(19) United States
(12) Patent Application Publication
(10) Pub. No.: US 2021/0060084 A1
(43) Pub. Date: Mar. 4, 2021

(54) AMNIOTIC FLUID FORMULATION FOR MODULATING IMMUNE RESPONSES

(71) Applicant: MAM Holdings of West Florida, L.L.C., Tarpon Springs, FL (US)

(72) Inventor: Carl Randall Harrell, Tarpon Springs, FL (US)

(21) Appl. No.: 16/999,259

(22) Filed: Aug. 21, 2020

Related U.S. Application Data
(60) Provisional application No. 62/893,671, filed on Aug. 29, 2019.

(51) Int. Cl. A61K 35/50; A61P 37/06

(52) U.S. Cl. CPC A61K 35/50; A61K 45/06; A61P 37/06

(57) ABSTRACT

Compositions and formulations of de-cellularized human amniotic fluid (D-HAF) and methods of use thereof are described. The compositions and formulations typically including over 300 human growth factors and stem cell derived exosomes can be used for therapeutic immunosuppression strategies useful in the treatment of inflammatory diseases or disorders, autoimmune diseases or disorders, inducing or increase graft tolerance, treating graft rejection, and treating allergies and other ailments particularly those involving eyes, joints, and the respiratory system with symptoms that can be reduced or ameliorated by regulating the activity of T cells (Th1, Th2, Th17, and/or Tregs), NK cells, antigen-presenting cells, or combinations thereof. Compositions and methods for balancing a T-helper cell profile and in particular for suppressing or reducing expansion of inflammatory Th1 and Th17 cells and/or promoting generation of immunosuppressive Tregs are provided.

### CROSS-REFERENCE TO RELATED APPLICATIONS

This application claims benefit of U.S. Provisional Application No. 62/893,671, filed Aug. 29, 2019, which is incorporated by reference in its entirety.

### FIELD OF THE INVENTION

This invention relates to compositions including sterile de-cellularized human amniotic fluid, and formulations and methods thereof for the treatment and/or prevention of one or more of neurodegenerative diseases, autoimmune diseases or disorders as well as acute and chronic inflammatory diseases.

### BACKGROUND OF THE INVENTION

Amniotic fluid (AF) is a complex and dynamic milieu that changes as pregnancy progresses. AF contains nutrients and growth factors that facilitate fetal growth, provides mechanical cushioning and antimicrobial effectors that protect the fetus, and allows assessment of fetal maturity and disease. AF contains a plethora of factors including carbohydrates, proteins and peptides, lipids, lactate, pyruvate, electrolytes, enzymes, and hormones. AF contains many important growth factors including epidermal growth factor (EGF), transforming growth factor alpha (TGF-α), transforming growth factor beta-1, insulin-like growth factor I (IGF-I), and erythropoietin (EPO).

Amniotic fluid stem cells (AFS) have shown a distinct secretory profile and significant regenerative potential in several preclinical models of disease. AFS are fetal mesenchymal progenitors with a distinct cardioprotective and angiogenic secretory profile that can be easily isolated, expanded, and cryopreserved, hence representing a suitable candidate for future clinical applications. Nevertheless, little is known about the detailed characterization of their secretome, which includes the entirety of growth factors and chemoattractant molecules produced by stem cells. It has been shown that AFS actively release extracellular vesicles (EV) endowed with significant paracrine potential and regenerative effect. EV are membrane-bound cellular components enriched with soluble, bioactive factors (proteins, lipids, etc.) and RNA (mainly regulatory microRNA—miRNA). They elicit a wide range of effects while mediating horizontal intercellular transfer of genetic information on the responder cell, consequently modulating its function. EV are secreted as microsized (microvesicles: 100-1,000 nm) and nanosized (exosomes: 30-150 nm) particles, thus acting as key biological effectors of paracrine signaling. AFS can be easily isolated, expanded, and cryopreserved, hence representing a suitable candidate for future clinical applications.

However, despite the therapeutic promises of the human amniotic fluid and human amniotic fluid stem cells, there remain few options to precisely modulate pro-inflammatory and/or suppressive immune responses. Identification and characterization of components of human amniotic fluid is important for understanding of the mechanisms of its regulation of immune functions and for the development of new therapies for the treatment of diseases and disorders related to these mechanisms.

Therefore, it is an object of the invention to provide compositions and pharmaceutical formulations of human amniotic fluid with the immunomodulatory functions.

It is another object of the invention to provide compositions that modulate pro-inflammatory immune response and/or pro-inflammatory cytokine production and methods of use thereof to reduce pro-inflammatory cell proliferation or activation such as Th1 and Th17 and production of associated cytokines.

It is another object of the invention to provide compositions and methods to inhibit or reduce recruitment of immune cells including neutrophils, macrophages, and monocytes or other cells involved in inflammatory response.

It is another object of the invention to provide compositions that modulate immune response via inducing and enhancing activity of Tregs and associated cytokine production such as IL-10.

It is another object of the invention to provide compositions and methods for treating inflammatory diseases or disorders, autoimmune diseases or disorders, inducing or increase graft tolerance, treating graft rejection, and treating allergies and other ailments particularly those involving eyes, joints, brain, and the respiratory system with symptoms that can be reduced or ameliorated by regulating the associated immune responses.

### SUMMARY OF THE INVENTION

A de-cellularized amniotic fluid (D-HAF), optionally including one or more pharmaceutically acceptable excipients, is devoid of amniotic cells, micronized amnion membrane and chorion membrane particles. It is sterilized without the use of ultraviolet radiation, heat or chemicals that can decrease the biological activity. The composition can be administered with other therapeutic, prophylactic or diagnostic agent, such as neuroprotective agents, antimicrobial agents, local anesthetics, antioxidants, anti-inflammatory agents, growth factors, immunosuppressant agents, anti-allergic agents, and combinations thereof to a subject in need thereof. In some embodiments, the D-HAF compositions include one or more exosomes generated ex vivo from mesenchymal stem cells, preferably amniotic fluid mesenchymal stem cells.

The compositions and formulations can be used in therapeutic immunosuppression strategies useful in the treatment of inflammatory diseases or disorders, autoimmune diseases or disorders, inducing or increase graft tolerance, treating graft rejection, and treating allergies and other ailments particularly those involving eyes, joints, brain, and the respiratory system with symptoms that can be reduced or ameliorated by regulating the activity of T cells (Th1, Th2, Th17, and/or Tregs), NK cells, antigen-presenting cells, or combinations thereof.

Methods of modulating an immune response in a subject in need thereof by administering an effective amount of the compositions to reduce activation, proliferation and/or generation of one or more pro-inflammatory cells, and/or enhance activation, proliferation and/or generation of one or more suppressive immune cells are provided. In some embodiments, the pro-inflammatory cells are T helper type 1 (Th1) cells, T helper type 17 (Th17) cells, or both. In further embodiments, the suppressive immune cells are regulatory T cells (Tregs). Methods are effective in treating one or more symptoms such as diseases or disorders such as inflammatory diseases, autoimmune disorders, and transplant rejection. In particular, the methods are effective for treating symptoms of one or more autoimmune disorders such as rheumatoid arthritis, systemic lupus erythematosus, alopecia areata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), IgA nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

Compositions and methods for increasing a ratio of the level of endogenous regulatory T (Treg) cells to the level of endogenous pro-inflammatory T cells such as Th1 and/or Th17 cells in a subject in need thereof for treating or alleviating one or more symptoms associated with an inflammatory disease, an autoimmune disorder, or transplant rejection are also described.

The methods can be employed to treat subjects with an inflammatory or autoimmune disease/disorder, in particular those associated with an elevated frequency and/or number of one or more pro-inflammatory cells, and/or reduced frequency and/or number of one or more suppressive immune cells. Typically, the methods include administering to the subject in need thereof an amount of the de-cellularized sterile amniotic fluid which is effective to reduce the frequency and/or number of Th1, Th17, or both; and/or to increase the frequency and/or number of Tregs. The compositions and formulations can be administered to a subject to effectively reduce Th1 and Th17 responses; or enhance regulatory T cell (Treg) responses, or reduce elevated levels of one or more cytokines selected from the group consisting of IL-1, IL-6, TNF-a, IL-17, IL21, and IL23.

The compositions and formulations can be administered to a subject to promote or increase a regulatory T cell response, proliferation of regulatory T cells, differentiation and effector function of regulatory T cells or survival of regulatory T cells in the subject. Methods of using the compositions and formulations to treat or inhibit one or more symptoms of an inflammatory response, to treat or inhibit one or more symptoms of an autoimmune disease, to reduce or inhibit transplant rejection or to treat one or more symptoms of graft versus host disease (GVHD) in a subject are provided.

Methods of using the compositions and formulations to treat or alleviate one or more symptoms of a stroke, a traumatic brain injury, a spinal cord injury, Post-Traumatic Stress syndrome, dementia, or a combination thereof, in a subject in need thereof are also provided.

### BRIEF DESCRIPTION OF THE DRAWINGS

FIGS. 1A-1E are bar graphs showing concentrations of pro-inflammatory IL-12 (FIG. 1A) in the supernatants of LPS-primed pbMNCs in the absence or presence of treatment with 4° C. or RT-stored de-cellularized amniotic fluid (Exo-d-MAPPS) or 4° C. or RT-stored raw amniotic fluid (AF), percentages of CD14+ cells (FIG. 1B), percentage of CD14+HLA-DR+ cells (FIG. 1C), percentage of CD14+TNF-α+ cells (FIG. 1D), and percentage of CD14+IL-10+ cells (FIG. 1E) in the pbMNCs primed with LPS with or without treatment with 4° C. or RT-stored de-cellularized amniotic fluid (Exo-d-MAPPS) or 4° C. or RT-stored raw amniotic fluid (AF). Values are mean±SEM; *p<0.05, **p<0.01, ***p<0.001.

FIGS. 2A-2G are bar graphs showing percentages of CD4+ T cells (FIG. 2A), percentages of IFN-γ-producing CD4+ T cells (FIG. 2B), percentages of IL-17-producing CD4+ T cells (FIG. 2C), percentages of Foxp3+ regulatory CD4+ cells (FIG. 2D), and percentages of IL-10+ regulatory CD4+ cells (FIG. 2E) in Con A-primed pbMNCs with or without treatment with 4° C. or RT-stored de-cellularized amniotic fluid (Exo-d-MAPPS) or 4° C. or RT-stored raw amniotic fluid (AF), and concentrations of pro-inflammatory IL-17 (FIG. 2F) and anti-inflammatory IL-10 (FIG. 2G) in the supernatants of ConA-primed pbMNCs with or without treatment with 4° C. or RT-stored de-cellularized amniotic fluid (Exo-d-MAPPS) or 4° C. or RT-stored raw amniotic fluid (AF). Data presented as mean±SEM; *p<0.05, **p<0.01, ***p<0.001.

FIGS. 3A-3F are bar graphs showing percentages of CD56+ cells (FIG. 3A), percentages of IFN-γ-producing CD56+ cells (FIG. 3B), percentages of IL-17-producing CD56+ cells (FIG. 3C), percentages of regulatory Foxp3+CD56+ cells (FIG. 3D), percentages of regulatory IL-10+CD56+ cells (FIG. 3E) in α-GalCer-primed pbMNCs with or without treatment with 4° C. or RT-stored de-cellularized amniotic fluid (Exo-d-MAPPS) or 4° C. or RT-stored raw amniotic fluid (AF); and concentrations of anti-inflammatory IL-10 in the supernatants of α-GalCer-primed pbMNCs with or without treatment with 4° C. or RT-stored de-cellularized amniotic fluid (Exo-d-MAPPS) or 4° C. or RT-stored raw amniotic fluid (AF) (FIG. 3F). Values are mean±SEM; *p<0.05, **p<0.01, ***p<0.001.

### DETAILED DESCRIPTION OF THE INVENTION

### I. Definitions

The term “Active Agent,” refers to a physiologically or pharmacologically active therapeutic, prophylactic or diagnostic agent that acts locally and/or systemically in the body. An active agent is a substance that is administered to an individual for the treatment (e.g., therapeutic agent), prevention (e.g., prophylactic agent), or diagnosis (e.g., diagnostic agent) of a disease or disorder. Active agents may also include materials that alleviate symptoms such as shortness of breath.

The phrase “therapeutically effective amount” refers to an amount of the therapeutic agent that produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. A prophylactic agent refers to an agent that may prevent a disorder, disease or condition.

The terms “immunologic”, “immunological” or “immune” response is the development of a beneficial humoral (antibody mediated) and/or a cellular (mediated by antigen-specific T cells or their secretion products) response directed against an immunogen in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The term “tissue repair”, refers to the restoration of tissue architecture and function after an injury in the context of the healing of damaged tissue. It encompasses cellular regeneration. Regeneration refers to a type of healing in which new growth restores portions of damaged tissue to an improved state, or to their normal state. Tissue regeneration can be initiated by stimulants in the formulations, and/or by stem cells introduced onto the damaged tissues.

The term “treating” refers to preventing or alleviating one or more symptoms of a disease, disorder or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The terms “enhance”, “increase”, “stimulate”, “promote”, “decrease”, “inhibit” or “reduce” are used relative to a control. Controls are known in the art. For example, an increase response in a subject or cell treated with a compound is compared to a response in subject or cell that is not treated with the compound.

The term “growth factors,” refers to a group of proteins or hormones that stimulate the cellular growth. Growth factors play an important role in promoting cellular differentiation and cell division, and they occur in a wide range of organisms.

The term “amniotic factor,” generally refers to molecules naturally present in the amniotic fluid. These include carbohydrates, proteins and peptides such as enzymes and hormones, lipids, metabolic substrates and products such as lactate and pyruvate, and electrolytes.

The term “biocompatible” or “biologically compatible,” generally refers to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

The term “biodegradable” as used herein means that the materials degrades or breaks down into its component subunits, or digestion, e.g., by a biochemical process, of the material into smaller (e.g., non-polymeric) subunits.

The term “pharmaceutically acceptable,” refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “molecular weight,” generally refers to the relative average chain length of the bulk polymer or protein, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

### II. Compositions

Formulations of de-cellularized human amniotic fluid are provided. The formulations include sterile de-cellularized human amniotic fluid devoid of cells and particulate matter via a series of centrifugation and filtration steps. The fluid formulations generally include exosomes derived from amniotic fluid stem cells. The concentrations of proteins, lipids, or any other molecules present in the de-cellularized human amniotic fluid are similar to that of the raw amniotic fluid. Typically, the de-cellularized amniotic fluid retains more than 80% of the amniotic proteins compared to the raw amniotic fluid. In some embodiments, D-HAF compositions retain most amniotic factors after short-term or long-term storage under temperature-controlled conditions either as a liquid or as lyophilized powder, for example, at least 50% of the total protein content compared to that of the fresh D-HAF, preferably more than 80%.

The compositions and formulations can be used for therapeutic immunosuppression strategies useful in the treatment of inflammatory diseases or disorders, autoimmune diseases or disorders, inducing or increase graft tolerance, treating graft rejection, and treating allergies and other ailments with symptoms that can be reduced or ameliorated by regulating the activity of T cells (Th1, Th17, and/or Tregs), NK cells, antigen-presenting cells, or combinations thereof. In some embodiments, the compositions and formulations can reduce autoantibodies (e.g., anti-dsDNA autoantibodies) production in a subject in need thereof. In some embodiments, the compositions can suppress or reduce expansion of inflammatory Th1 and Th17 cells and/or promote generation of immunosuppressive Tregs. In preferred embodiments, the compositions can induce, increase, or enhance a functionally robust induced CD4 Treg population (e.g., Foxp3+ Treg) in vivo or ex vivo.

### A. Amniotic Fluid

Amniotic fluid (“AF”) contains nutrients and growth factors that facilitate fetal growth, provides mechanical cushioning and antimicrobial effectors that protect the fetus, and allows assessment of fetal maturity and disease. AF typically contains mixtures of growth factors, pro-inflammatory cytokines and anti-inflammatory cytokines, as well as a variety of macromolecules including carbohydrates, proteins and peptides, lipids, lactate, pyruvate, electrolytes, enzymes, and hormones.

### 1. Growth Factors, Cytokines and Other Molecules

Growth factors and their receptors control a wide range of biological functions, regulating cellular proliferation, survival, migration and differentiation. Growth factors found in AF play a critical role in fetal growth and development.

Some of the growth factors that have been identified in AF includes such as epidermal growth factor (EGF), insulin-like growth factor I (IGF-I), vascular endothelial growth factor A (VEGF-α), tumor necrosis factor A (TNF-α), hepatocyte growth factor (HGF), fibroblast growth factor 7 (FGF7), matrix metallopeptidase (MMP-9), granulocyte-colony stimulating factor (GCSF), matrix metalloproteinase-7 (MMP-7), matrix metalloproteinase-7 (MMP-13), transforming growth factor alpha (TGF-α), transforming growth factor beta (TGF-β), fibroblast growth factor 4 (FGF-4), endocrine gland-derived vascular endothelial growth factor (EG-VEGF), interleukin 8 (IL-8), fibroblast growth factor 21 (FGF-21), angiopoietin-2 (ANG2), Glial cell-derived neurotrophic factor (GDNF), fibroblast growth factor 19 (FGF-19), TIMP metallopeptidase inhibitor 2 (TIMP-2), angiopoietin-1 (ANG-1), Transforming growth factor beta 1(TGFβ1), macrophage colony-stimulating factor (M-CSF), angiotensinogen, platelet derived growth factor-AA (PDGF-AA), and stem cell factor (SCF).

Epidermal growth factor (EGF) is a small polypeptide hormone with mitogenic properties in vivo and in vitro. EGF elicits biologic responses by binding to a cell surface receptor which is a transmembrane glycoprotein containing a cytoplasmic protein tyrosine kinase. EGF responses are mediated by ligand binding and activation of this intrinsic protein kinase. The receptor can be phosphorylated by other protein kinases, and this may regulate receptor function. Stimulation of the receptor tyrosine kinase activity by ligand binding must regulate the activity of an as yet undefined molecule(s) responsible for transmitting a mitogenic signal to the nucleus (Todderud G, et al., Biofactors. 1989, 2(1):11-5).

Vascular endothelial growth factor (VEGF), also known as vascular permeability factor (VPF), was originally described as an endothelial cell-specific mitogen. VEGF is produced by many cell types including tumor cells, macrophages, platelets, keratinocytes, and renal mesangial cells. The activities of VEGF are not limited to the vascular system; VEGF plays a role in normal physiological functions such as bone formation, hematopoiesis, wound healing, and development (Duffy A M et al., In: Madame Curie Bioscience Database [Internet]. Austin (TX): Landes Bioscience (2000)).

TGF-α has a structure similar to EGF and binds to the same receptor. The amnion cells of the umbilical cord express EGF, TGF-α, and the functional EGF/TGF-α receptor, suggesting the possibility of a regulating role of the amnion in fetal growth and development. EGF and TGF-α have also been shown to stimulate the production of surfactant components. TGFβ1 is believed to induce terminal differentiation of intestinal epithelial cells and to accelerate the rate of healing of intestinal wounds by stimulating cell migration. TGFβ1 may also stimulate IgA production. VEGF-A is a signal protein that stimulates vasculogenesis and angiogenesis (Hoeben Am, et al., Pharmacol Rev 2004, 56:549-580).

Transforming growth factor-beta (TGF-β) is a multifunctional peptide that controls proliferation, differentiation, and other functions in many cell types. Many cells synthesize TGF-beta and essentially all of them have specific receptors for this peptide. TGF-beta regulates the actions of many other peptide growth factors and determines a positive or negative direction of their effects (Sporn M B, et al., Science 1986, 233(4763) 532-534).

Hepatocyte growth factor (HGF), the ligand for the receptor tyrosine kinase encoded by the c-Met proto-oncogene, is a multidomain protein structurally related to the pro-enzyme plasminogen and with major roles in development, tissue regeneration and cancer. A recent study showed its immunomodulation potential of amniotic fluid stem cells (Maraldi T, et al. Stem Cells Transl Med, 4(6):539-47 (2015)).

Fibroblast growth factors (FGFs) that signal through FGF receptors (FGFRs) regulate a broad spectrum of biological functions, including cellular proliferation, survival, migration, and differentiation. The FGF signal pathways are the RAS/MAP kinase pathway, PI3 kinase/AKT pathway, and PLCγ pathway, among which the RAS/MAP kinase pathway is known to be predominant. Several studies have recently implicated the in vitro biological functions of FGFs for tissue regeneration. Many current applications of FGF are in regeneration of tissues, including skin, blood vessel, muscle, adipose, tendon/ligament, cartilage, bone, tooth, and nerve tissues (Yun Y R, et al., J Tissue Eng 2010: 1(1)).

Matrix metalloproteinases (MMPs), also called matrixins, function in the extracellular environment of cells and degrade both matrix and non-matrix proteins. They play central roles in morphogenesis, wound healing, tissue repair and remodeling in response to injury, e.g. after myocardial infarction, and in progression of diseases such as atheroma, arthritis, cancer and chronic tissue ulcers. They are multi-domain proteins and their activities are regulated by tissue inhibitors of metalloproteinases (TIMPs) (Nagase H, et al., Cardiovascular Research, European Society of Cardiology, 562-573 (2006)).

Amniotic fluid also contains many pro- and anti-inflammatory cytokines. Pro- and anti-inflammatory cytokines play important immunoregulatory roles. Inflammation is characterized by interplay between pro- and anti-inflammatory cytokines. Cytokines are commonly classified in one or the other category: interleukin-1 (IL-1), tumor necrosis factor (TNF), gamma-interferon (IFN-gamma), IL-12, IL-18 and granulocyte-macrophage colony stimulating factor are well characterized as pro-inflammatory cytokines whereas IL4, IL-10, IL-13, IFN-alpha and transforming growth factor-beta are recognized as anti-inflammatory cytokines.

Exemplary pro-inflammatory cytokines include Eotaxin-2 (CCL24), interleukin 6 (IL-6), pulmonary and activation-regulated chemokine PARC or chemokine (C-C motif) ligand 18 (CCL18), total GRO which consisted of three subunits GROα/CXCL1, GROβ/CXCL2, and GROγ/CXCL3, expression of the neutrophil-activating CXC chemokine (ENA-78/CXCL-5), chemokine (C-C motif) ligand 21 (CCL21 or 6Ckine), macrophage inflammatory protein 3 alpha (MIP-3α or CCL20), monokine induced by gamma (MIG orCXCL-9), MIP-1α, chemokine (C-C motif) ligand 5 (CCL-5), also known as RANTES (regulated on activation, normal T cell expressed and secreted), Interleukin-1 alpha (IL-1α), macrophage inflammatory protein-1β (MIP-1β or CCL4), tumor necrosis factor (TNFα) and monocyte chemotactic protein 2 (MCP-2 or CCL8).

Exemplary anti-inflammatory cytokines include the anti-inflammatory factors include interleukin 8 (IL-8), interleukin 13 (IL-13), interleukin 27 (IL-27), cytotoxic T-lymphocyte-associated protein 4 (CTLA-4), vascular endothelial growth factor D (VEGF-D), interleukin-1 receptor antagonist (IL-1Ra), transforming growth factor beta 1 (TG931), interleukin 5 (IL-5) and interleukin 21 (IL-21).

### 2. Stem Cell Exosomes

Typically, the disclosed de-cellularized amniotic fluid compositions include stem cell exosomes such as those derived from amniotic mesenchymal stem cells (MSCs). MSCs are fibroblast-like multipotent cells capable to self-renew and, under appropriate culture conditions, differentiate into the cells of mesodermal, endodermal and ectodermal lineage (Friedenstein A J, et al., Transplantation. 1968;6:230-247; Bieback K, et al., Stem Cells. 2004;22:625-634; Yanez R, et al., Stem Cells. 2006;24:2582-2591; Anversa P, et al., Eur J Clin Invest. 2012;42:900-913). MSCs are present in virtually all postnatal tissues and organs and after isolation (from the bone marrow, umbilical cord blood, placenta, adipose tissue, amniotic fluid, Wharton's jelly) may be easily propagated to reach appropriate cell number for autologous or allogeneic transplantation (Bieback K, et al., Stem Cells. 2004;22:625-634; Yanez R, et al., Stem Cells. 2006;24:2582-2591). Therefore, large number of experimental and clinical studies indicated that MSCs could be considered as new remedy in cell-based therapy of degenerative diseases (Volarevic V, et al., Br Med Bull. 2011;99:155-168).

Additionally, MSCs are able to modulate phenotype of immune cells and may suppress detrimental, local and systemic immune response (Harrell CR, et al., Adv Exp Med Biol. 2019 Jun. 8. doi: 10.1007/5584_2018_306). In juxtacrine, cell to cell contact-dependent manner and paracrine manner (through the production of soluble mediators), MSCs alter the function of all immune cells (macrophages, dendritic cells (DCs), natural killer (NK), natural killer T cells (NKT), T and B lymphocytes) that have essential role in the pathogenesis of autoimmune, acute and chronic inflammatory diseases (Simovic Markovic B, et al., Stem Cells Int. 2017;2017:1315378; Gazdic M, et al., J Tissue Eng Regen Med. 2018;12:e1173-e1185; Simovic Markovic B, et al., Stem Cells Int. 2016;2016:2640746).

Production of immunoregulatory factors in MSCs and their capacity for immunosuppression was identified by Haynesworth and coworkers (Haynesworth S E, et al., J Cell Physiol. 1996;166:585-92). They reported that MSCs produce and release a broad repertoire of growth factors, chemokines, and cytokines that modulate production of inflammatory cytokines in immune cells affecting their functional properties. Additionally, further studies revealed that MSC-sourced factors promote neo-angiogenesis, reduce apoptosis and enhance survival of parenchymal cells, regulate remodeling of extracellular matrix and prevent fibrosis in injured tissues, enabling enhanced tissue repair and regeneration (Caplan A I, et al., J Cell Biochem. 2006;98:1076-84; Moravej A, et al., Immunol Invest. 2017;46:80-96).

Among immunomodulatory factors, MSCs produce transforming growth factor-β (TGF-β), hepatic growth factor (HGF), nitric oxide (NO), indolamine 2,3-dioxygenase (IDO), IL-10, IL-6, leukocyte inhibitory factor (LIF), IL-1 receptor antagonist (IL-1Ra), tumor necrosis factor a-stimulated gene 6 (TSG-6), human leukocyte antigen-G (HLA-G), hemeoxygenase-1 (H0-1), and prostaglandin E2 (PGE2) (Harrell CR, et al., Adv Exp Med Biol. 2019 Jun. 8. doi: 10.1007/5584_2018_306; Volarevic V, et al., Biofactors. 2017;43:633-644; Harrell C R, et al., Cells. 2019;8(5). pii: E467. doi: 10.3390/cells8050467). Therefore, local as well as systemic administration of MSCs-sourced secretome, including MSC-derived conditioned medium (MSC-CM) and MSC-derived exosomes (MSC-Exos), showed beneficial effects similar to those observed after transplantation of MSCs. Due to their nano-size dimension, MSC-Exos easily penetrate through the tissue and in paracrine and endocrine manner, deliver MSC-sourced factors to the target immune cells modulating their function (Mohammadipoor A, et al., Respir Res. 2018;19:218).

Several lines of evidence suggested that MSCs derived from amniotic fluid (AF-MSCs) had superior cell biological properties than MSCs derived from bone marrow (BM-MSCs) (Hass R, et al., Cell Commun Signal. 2011;9:12; Kil K, et al., Int J Pediatr Otorhinolaryngol. 2016;91:72-81; Cho JS, et al., Yonsei Med J. 2018;59:406-415; Jiang H, et al., Sci Rep. 2017;7:41837). Roubelakis and colleagues revealed that AF-MSCs have 78 unique proteins which are responsible for their increased proliferation rate and plasticity (Roubelakis M G, et al., Stem Cells Dev. 2007;16:931-952). Furthermore, AF-MSCs more efficiently suppressed detrimental T cell-driven immune response than BM-MSCs (Mareschi K, et al., Exp Hematol. 2016;44:138-150.e1).

Exosomes derived from AF-MSCs represent a major therapeutic option in regenerative medicine, capable of inducing damaged tissue repair, and exerting immunomodulatory effects on the differentiation, activation and function of different lymphocytes. They are also suitable candidates for allogeneic therapy due to their low immunogenicity.

The sterile de-cellularized amniotic fluid typically includes exosomes derived from amniotic fluid stem cells (AFSC). Accordingly, in one aspect, the D-HAF including exosomes derived from AFSC or from amniotic fluid mesenchymal stem cells is effective for treating and/or alleviating one or more symptoms of the targeted diseases.

In some embodiments, exosomes derived from MSCs contain one or more of immunomodulatory factors of MSCs including transforming growth factor-β (TGF-β), hepatic growth factor (HGF), nitric oxide (NO), indolamine 2,3-dioxygenase (IDO), IL-10, IL-6, leukocyte inhibitory factor (LIF), IL-1 receptor antagonist (IL-1Ra), tumor necrosis factor a-stimulated gene 6 (TSG-6), human leukocyte antigen-G (HLA-G), hemeoxygenase-1 (HO-1), and prostaglandin E2 (PGE2). In some embodiments, AFSC-derived exosomes present different molecules than those non-exosome associated soluble factors in the de-cellularized amniotic fluid, some of them involved in immunomodulation.

Amniotic fluid MSCs are broadly multipotent, can typically be expanded in culture, and can be easily cryopreserved in cellular banks. Thus, in some embodiments, MSCs separated or isolated from amniotic fluid are cultured for generating further exosomes. In some embodiments, amniotic fluid MSCs express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. Further embodiments encompass culturing MSCs that do not express substantial levels of HLA-DR, CD117, and CD45. The phenotype and homogeneity of the amniotic fluid MSCs can be analyzed by techniques such as fluorescence-activated cell sorting (FACS) following standard protocol with a panel of mouse monoclonal antibodies, for example, including one or more of CD14, CD29, CD44, CD45, CD90, and CD105 (BD Pharmingen; San Jose, Calif.) on an cell analyzer.

In further embodiments, MSCs are derived from a source selected from bone marrow, adipose tissue, amniotic fluid, umbilical cord blood, placental tissue, differentiated embryonic stem cells, and differentiated progenitor cells. In preferred embodiments, MSCs are amniotic fluid MSCs.

Exemplary techniques for generating exosomes include liposome stimulation using one or more stimulant liposomes such as neutral or cationic liposomes (Emam S E et al., Biol Pharm Bull. 2018;41(5):733-742),

Exosomes can be isolated by many known techniques including reagent-enhanced centrifugation such as Total Exosome Isolation Reagent (Invitrogen, Thermo Fisher Scientific).

Accordingly, in some aspects, the amniotic fluid formulations further include exosomes generated ex vivo from amniotic fluid MSCs, or derived from MSCs of other sources. In some embodiments, exosomes generated ex vivo are enriched with one or more additional proteins, nucleic acids, carbohydrates, or other therapeutic agents.

### 3. Sources of Amniotic Fluid Formulations

The amniotic fluid formulations are prepared from sterile human amniotic fluid obtained from a pregnant woman. Suitable sources, e.g. of human AF, include AF that is obtained from patients who are undergoing amniocentesis, patients who are undergoing a Caesarean section delivery, and patients undergoing normal delivery using a specially designed receptacle to collect the fluid after rupture of membranes.

The de-cellularized human amniotic fluid (D-HAF) formulations can be stored for long periods of time, allowing for a broad range of application methods, including distribution and storage as aerosols, solutions, powders, etc. In some embodiments, the sterile D-HAF is refrigerated at about 1° C. to about 10° C. for long-term storage. In a further embodiment, the sterile D-HAF is refrigerated at 4° C. for up to 12 months and more. In yet another embodiment, the sterile D-HAF is kept at room temperature for 1 month, 2 months, 3 months, 4 months, 5 months, or up to 12 months. Preferably, the long-term storage does not reduce the quantity of the total soluble proteins or factors present in the D-HAF. For some embodiments, the total soluble proteins retained after long-term storage in refrigerated conditions or room temperature is about 10%, 20%, 30%, 40%, 50%, 60%, 70%, 80%, or 90% of the fresh D-HAF.

D-HAF formulations containing amniotic factors can be supplied as a clear one-part solution in a suitable container for storage at room temperature, 4° C., or for storage at −20° C., or at −80° C. For example, liquid formulations in prefilled aliquots can be suitable for storage at 1-5° C., or for storage at −20° C., or at −80° C. The liquid formulation can be suitable for topical application in a nebulizer or an inhaler. In other embodiments, the fluid can be supplied as a kit that can be stored at room temperature, 4° C., at −20° C., or at −80° C. until needed.

In some embodiments, D-HAF formulations use a final filtration through 0.2 μm is necessary to get the best sterility assurance level and produce a sterile amniotic fluid without any irradiation. In some embodiments, D-HAF formulations have a 10⁻⁶ sterility assurance level without irradiation. In other embodiments, lyophilisate derived from amniotic fluid through lyophilization may be irradiated by e-beam irradiation or gamma ray irradiation to add another guarantee for the final sterility of the powder.

In some embodiments, D-HAF formulations are synthesized amniotic fluid to include all the known amniotic factors for the same therapeutic, and/or prophylactic properties in treating one or more diseases or disorders, autoimmune diseases or disorders, inducing or increase graft tolerance, treating graft rejection, and treating allergies and other ailments particularly those involving eyes, joints, brain, and the respiratory system.

### B. Additional Therapeutic, Prophylactic or Diagnostic Agents

In addition to the amniotic fluid component, the formulation can contain one or more additional therapeutic, diagnostic, and/or prophylactic agents. In some embodiments, the composition may contain one or more additional compounds to relief symptoms such as inflammation, or shortness of breath. Representative therapeutic (including prodrugs), prophylactic or diagnostic agents can be peptides, proteins, carbohydrates, nucleotides or oligonucleotides, small molecules, or combinations thereof. Non-limiting examples include neuroprotective agents, corticosteroids, antimicrobial agents, analgesics, local anesthetics, anti-inflammatory agents, immunosuppres sant agents, anti-angiogenesis agents, anti-allergenic agents, enzyme cofactors, essential nutrients and growth factors.

The active agents can be a small molecule active agent or a biomolecule, such as an enzyme or protein, polypeptide, or nucleic acid. Suitable small molecule active agents include organic and organometallic compounds. In some instances, the small molecule active agent has a molecular weight of less than about 2000 g/mol, more preferably less than about 1500 g/mol, most preferably less than about 1200 g/mol. The small molecule active agent can be a hydrophilic, hydrophobic, or amphiphilic compound.

In some cases, one or more additional active agents may be encapsulated in, dispersed in, or otherwise associated with particles in the formulation. In certain embodiments, one or more additional active agents may also be dissolved or suspended in the pharmaceutically acceptable carrier.

In the case of pharmaceutical compositions for the treatment of lung diseases, the formulation may contain one or more therapeutic agents to treat, prevent or diagnose a disease or disorder of the lung. Non-limiting examples of therapeutic agents include corticosteroids, anti-angiogenesis agents, antibiotics, antioxidants, anti-viral agents, anti-fungal agents, anti-inflammatory agents, growth factors, immunosuppressant agents, anti-allergic agents, and combinations thereof.

The amount of a second therapeutic generally depends on the severity of lung disorders to be treated. Specific dosages can be readily determined by those of skill in the art. See Ansel, Howard C. et al. Pharmaceutical Dosage Forms and Drug Delivery Systems (6^{th} ed.) Williams and Wilkins, Malvern, Pa. (1995). Alternatively, the amniotic formulation can be used in combination with cell delivery, for example, the delivery of stem cells, pluripotent cells, somatic cells, or combinations thereof.

In other embodiments, one or more agents include neuroprotective agents, corticosteroids, antimicrobial agents, analgesics, local anesthetics, anti-inflammatory agents, immunosuppressant agents, anti-angiogenesis agents, anti-allergenic agents, enzyme cofactors, essential nutrients and growth factors are administered prior to, in conjunction with, subsequent to, or alternation with treatment with the amniotic fluid formulation.

The additive drug may be present in its neutral form, or in the form of a pharmaceutically acceptable salt. In some cases, it may be desirable to prepare a formulation containing a salt of an active agent due to one or more of the salt's advantageous physical properties, such as enhanced stability or a desirable solubility or dissolution profile.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, Md., 2000, p. 704.

In some cases, the additional agent is a diagnostic agent imaging or otherwise assessing the site of application. Exemplary diagnostic agents include paramagnetic molecules, fluorescent compounds, magnetic molecules, and radionuclides, x-ray imaging agents, and contrast media. These may also be ligands or antibodies which are labelled with the foregoing or bind to labelled ligands or antibodies which are detectable by methods known to those skilled in the art.

In certain embodiments, the pharmaceutical composition contains one or more local anesthetics. Representative local anesthetics include tetracaine, lidocaine, amethocaine, proparacaine, lignocaine, and bupivacaine. In some cases, one or more additional agents, such as a hyaluronidase enzyme, is also added to the formulation to accelerate and improves dispersal of the local anesthetic.

In some embodiments, the amniotic fluid formulation is used in combination with oxygen therapy.

### 1. Antigens

The amniotic fluid compositions can be used for treating, preventing and/or alleviating one or more symptoms associated with an abnormal/excessive immune response, such as an auto-immune disease, a response to a vaccine or a tissue/cell transplantation. In some case, it is highly desirable to be provided with a therapeutic formulation capable of inducing a state of anergy or immune tolerance by increasing the total number or proliferation of regulatory T cells (such as Treg), or reducing the total number or proliferation of the pro-inflammatory T cells (such as Th1 and Th17), or increase the ratio of the level of regulatory T cells (such as Treg) to pro-inflammatory T cells (such as Th1 and Th17). In further embodiments, amniotic fluid formulations are provided in combination with one or more autoimmune disease antigens.

Autoimmune disease antigens include, but are not limited to, degenerative disease antigen, atopic disease antigen, autoimmune disease antigen, alloantigen, xenoantigen, allergens, drugs include addictive substances such as nicotine, metabolic disease enzymes, enzymatic products, anti-drug antibody, and vector antigens. Self-antigens include Rh blood group antigens, I antigen, Platelet integrin GpIIb:IIIa, noncollagenous domain of basement membrane collagen type IV, epidermal cadherin, streptococcal cell-wall antigens, antibodies cross-reacting with cardiac muscle, Rheumatoid factor IgG complexes with or without Hep C antigens, DNA, histones, ribosomes, snRNP, scRNP, pancreatic beta-cell antigen, synovial joint antigen, myelin basic protein, proteolipid protein, myelin oligodendrocyte glycoprotein, and thyroid peroxidase.

### 2. Neuroprotective Agents

In some embodiments, amniotic fluid formulations are used in combination with one or more agents for the treatment of neurodegenerative diseases, neurological dysfunction, and/or neuroprotective agents. In further embodiments, the formulations are administered with one or more agents suitable for traumatic brain injuries, dementia, or PTSD.

Active agents for the treatment of neurodegenerative diseases are well known in the art and can vary based on the symptoms and disease to be treated. For example, conventional treatment for Parkinson's disease can include levodopa (usually combined with a dopa decarboxylase inhibitor or COMT inhibitor), a dopamine agonist, or an MAO-B inhibitor.

Treatment for Huntington's disease can include a dopamine blocker to help reduce abnormal behaviors and movements, or a drug such as amantadine and tetrabenazine to control movement, etc. Other drugs that help to reduce chorea include neuroleptics and benzodiazepines. Compounds such as amantadine or remacemide have shown preliminary positive results. Hypokinesia and rigidity, especially in juvenile cases, can be treated with antiparkinsonian drugs, and myoclonic hyperkinesia can be treated with valproic acid. Psychiatric symptoms can be treated with medications similar to those used in the general population. Selective serotonin reuptake inhibitors and mirtazapine have been recommended for depression, while atypical antipsychotic drugs are recommended for psychosis and behavioral problems.

Riluzole (RILUTEK®) (2-amino-6-(trifluoromethoxy) benzothiazole), an antiexcitotoxin, has yielded improved survival time in subjects with ALS. Other medications, most used off-label, and interventions can reduce symptoms due to ALS. Some treatments improve quality of life and a few appear to extend life. Common ALS-related therapies are reviewed in Gordon, Aging and Disease, 4(5):295-310 (2013), see, e.g., Table 1 therein. A number of other agents have been tested in one or more clinical trials with efficacies ranging from non-efficacious to promising. Exemplary agents are reviewed in Carlesi, et al., Archives Italiennes de Biologie, 149:151-167 (2011). For example, therapies may include an agent that reduces excitotoxicity such as talampanel (8-methyl-7H-1,3-dioxolo(2,3)benzodiazepine), a cephalosporin such as ceftriaxone, or memantine; an agent that reduces oxidative stress such as coenzyme Q10, manganoporphyrins, KNS-760704 [(6R)-4,5,6,7-tetrahydro-N6-propyl-2,6-benzothiazole-diamine dihydrochloride, RPPX], or edaravone (3-methyl-1-phenyl-2-pyrazolin-5-one, MCI-186); an agent that reduces apoptosis such as histone deacetylase (HDAC) inhibitors including valproic acid, TCH346 (Dibenzo(b,f)oxepin-10-ylmethyl-methylprop-2-ynylamine), minocycline, or tauroursodeoxycholic Acid (TUDCA); an agent that reduces neuroinflammation such as thalidomide and celastol; a neurotropic agent such as insulin-like growth factor 1 (IGF-1) or vascular endothelial growth factor (VEGF); a heat shock protein inducer such as arimoclomol; or an autophagy inducer such as rapamycin or lithium.

Treatment for Alzheimer's Disease can include, for example, an acetylcholinesterase inhibitor such as tacrine, rivastigmine, galantamine or donepezil; an NMDA receptor antagonist such as memantine; or an antipsychotic drug.

Treatment for Dementia with Lewy Bodies can include, for example, acetylcholinesterase inhibitors such as tacrine, rivastigmine, galantamine or donepezil; the N-methyl d-aspartate receptor antagonist memantine; dopaminergic therapy, for example, levodopa or selegiline; antipsychotics such as olanzapine or clozapine; REM disorder therapies such as clonazepam, melatonin, or quetiapine; anti-depression and antianxiety therapies such as selective serotonin reuptake inhibitors (citalopram, escitalopram, sertraline, paroxetine, etc.) or serotonin and noradrenaline reuptake inhibitors (venlafaxine, mirtazapine, and bupropion) (see, e.g., Macijauskiene, et al., Medicina (Kaunas), 48(1):1-8 (2012)).

Exemplary neuroprotective agents are also known in the art in include, for example, glutamate antagonists, antioxidants, and NMDA receptor stimulants. Other neuroprotective agents and treatments include caspase inhibitors, trophic factors, anti-protein aggregation agents, therapeutic hypothermia, and erythropoietin.

Other common active agents for treating neurological dysfunction include amantadine and anticholinergics for treating motor symptoms, clozapine for treating psychosis, cholinesterase inhibitors for treating dementia, and modafinil for treating daytime sleepiness.

### 3. Antimicrobial Agents

In some embodiments, amniotic fluid formulations are used in combination with one or more antimicrobial agents. An antimicrobial agent is a substance that kills or inhibits the growth of microbes such as bacteria, fungi, viruses, or parasites. Antimicrobial agents include antiviral agents, antibacterial agents, antiparasitic agents, and anti-fungal agents. Representative antiviral agents include ganciclovir and acyclovir. Representative antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines.

Other exemplary antimicrobial agents include iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

### 4. Local Anesthetics

In some embodiments, amniotic fluid formulations are used in combination with one or more local anesthetics. A local anesthetic is a substance that causes reversible local anesthesia and has the effect of loss of the sensation of pain. Non-limiting examples of local anesthetics include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and any combination thereof. In other aspects of this embodiment, the amniotic fluid formulations comprises an anesthetic agent in an amount of, e.g., about 10 mg, about 50 mg, about 100 mg, about 200 mg, or more than 200 mg. The concentration of local anesthetics in the compositions can be therapeutically effective meaning the concentration is adequate to provide a therapeutic benefit without inflicting harm to the patient.

### 5. Anti-inflammatory Agents

In some embodiments, amniotic fluid formulations are used in combination with one or more anti-inflammatory agents. Anti-inflammatory agents reduce inflammation and include steroidal and non-steroidal drugs. Suitable steroidal active agents include glucocorticoids, progestins, mineralocorticoids, and corticosteroids. In some embodiments, amniotic fluid formulations are used in combination with one or more corticosteroids.

Exemplary anti-inflammatory agents include triamcinolone acetonide, fluocinolone acetonide, methylprednisolone, prednisolone, dexamethasone, loteprendol, fluorometholone, ibuprofen, aspirin, and naproxen. Exemplary immune-modulating drugs include cyclosporine, tacrolimus and rapamycin. Exemplary non-steroidal anti-inflammatory drugs (NSAIDs) include mefenamic acid, aspirin, Diflunisal, Salsalate, Ibuprofen, Naproxen, Fenoprofen, Ketoprofen, Deacketoprofen, Flurbiprofen, Oxaprozin, Loxoprofen, Indomethacin, Sulindac, Etodolac, Ketorolac, Diclofenac, Nabumetone, Piroxicam, Meloxicam, Tenoxicam, Droxicam, Lornoxicam, Isoxicam, Meclofenamic acid, Flufenamic acid, Tolfenamic acid, elecoxib, Rofecoxib, Valdecoxib, Parecoxib, Lumiracoxib, Etoricoxib, Firocoxib, Sulphonanilides, Nimesulide, Niflumic acid, and Licofelone.

In some embodiments, anti-inflammatory agents are anti-inflammatory cytokines. Exemplary cytokines are IL-10, TGF-β and IL-35. Anti-inflammatory cytokines in the context of biomaterial implant, skin grafts, and hair grafts are cytokine that induce an anti-inflammatory immune environment or suppress inflammatory immune environment. Activation of regulatory T cells, Tregs, is involved in the prevention of rejection, the induction and maintenance of peripheral tolerance of the allograft. Th17 cells are a subset of T helper cells which is characterized by the production of IL-17. Th17 cells have been suggested to play a role in allograft rejection. In some embodiments, cytokines to be added to the amniotic fluid formulations are those that induce Tregs activation (e.g. IL-25) and suppress Th17 activation (e.g. IL-10) for minimizing rejection.

### 6. Cells

In some embodiments, the amniotic fluid formulation further comprises at least one eukaryotic cell type. Some exemplary eukaryotic cell types include stem cells, immune cells such as T lymphocytes, B lymphocytes, natural killer cells, and dendritic cells, or combinations thereof.

In some embodiments, the stem cells are mesenchymal stem cells. Functional characteristics of mesenchymal stem cells that may benefit wound healing include their ability to migrate to the site of injury or inflammation, participate in regeneration of damaged tissues, stimulate proliferation and differentiation of resident progenitor cells, promote recovery of injured cells through growth factor secretion and matrix remodeling, and exert unique immunomodulatory and anti-inflammatory effects.

In certain embodiments, the mesenchymal stem cells protect target/injured tissue through suppression of pro-inflammatory cytokines, and through triggering production of reparative growth factors.

### 7. Other Agents

In some embodiments, amniotic fluid formulations are used in combination with one or more growth factors. Growth factor, also known as a cytokine, refers to a protein capable of stimulating cellular growth, proliferation, and/or cellular differentiation. Non-limiting examples of growth factors include transforming growth factor beta (TGF-β), transforming growth factor alpha (TGF-α), granulocyte-colony stimulating factor (GCSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF8), growth differentiation factor-9 (GDF9), acidic fibroblast growth factor (aFGF or FGF-1), basic fibroblast growth factor (bFGF or FGF-2), epidermal growth factor (EGF), vascular endothelial growth factor (VEGF)and hepatocyte growth factor (HGF).

In some embodiments, the formulation can include antibodies, including, for example, daclizumab, bevacizumab (AVASTIN®), ranibizumab (LUCENTIS®), basiliximab, ranibizumab, and pegaptanib sodium or peptides like SN50, and antagonists of NF.

In further embodiments, the formulation can include oligonucleotides. Representative oligonucleotides include siRNAs, microRNAs, DNA, and RNA. Oligonucleotides can be used as gene therapy complementing the efficacy of the amniotic fluid formulations.

In some embodiments, the amniotic fluid formulation further comprises one or more enzyme cofactors, and/or one or more essential nutrients. Exemplary cofactors include vitamin C, biotin, vitamin E, and vitamin K. Exemplary essential nutrients are amino acids, fatty acids, etc.

### C. Excipients, Delivery Vehicles and Devices

Formulations and pharmaceutical compositions containing an effective amount of the D-HAF in a pharmaceutical carrier appropriate for administration to an individual in need thereof to treat one or more symptoms of inflammatory diseases, autoimmune diseases, or neurodegenerative diseases are provided.

The formulations can be designed for administration parenterally or enterally. It can also be administered topically (e.g., to a mucosal surface such as the mouth, lungs, intranasal, intravaginally, etc.). The compositions are designed to be administered locally or systemically.

The term “parenteral administration”, means administration by any method other than through the digestive tract or non-invasive topical or regional routes. For example, parenteral administration may include administration to a patient via intravenous, intradermal, intraperitoneal, intrapleural, intratracheal, intraarticular, intrathecal, intramuscular, subcutaneous, subjunctival, injection, and/or infusion.

The term “topical administration”, means the non-invasive administration to the skin, orifices, or mucosa. Topical administrations can be administered locally, i.e., they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, pulmonary, and rectal administration.

The term “enteral administration”, means administration via absorption through the gastrointestinal tract. Enteral administration can include oral and sublingual administration, gastric administration, or rectal administration.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, Md., 2000, p. 704.

Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions. D-HAF can be formulated for storage as a fluid or solid (i.e., powder). In preferred embodiments, DHAF is formulated for storage as a liquid (i.e., above freezing temperatures).

### 1. Solutions, Emulsions and Suspensions

Numerous formulations are known and available. Solutions can be the sterile filtered amniotic fluid, concentrated or diluted with water, buffered saline, or an equivalent. Emulsions are generally dispersions of oily droplets in an aqueous phase. There should be no evidence of breaking or coalescence. Suspensions contain solid particles dispersed in a liquid vehicle; they must be homogeneous when shaken gently and remain sufficiently dispersed to enable the correct dose to be removed from the container. Sediment may occur, but this should disperse readily when the container is shaken, and the size of the dispersed particles should be controlled. The active ingredient and any other suspended material must be reduced to a particle size small enough to be aerosolized and to prevent irritation and damage to the lining of the lungs. They may contain suitable additives, such as antimicrobial agents, antioxidants, and stabilizing agents.

When the solution is dispensed in a multidose container that is to be used over a period of time longer than 24 hours, a preservative must be added to ensure microbiologic safety over the period of use.

Formulations should be prepared depending on the intended use of the D-HAF and are well-known to those skilled in the art.

For example, for pulmonary applications, the pH of the formulations should be ideally equivalent to that of linings of the lung, which may vary depending on the precise location and the severity of the disease. However, the decision to add a buffering agent should be based on stability considerations. The pH selected should be the optimum for both stability of the active pharmaceutical ingredient and physiological tolerance. If a buffer system is used, it must not cause precipitation or deterioration of the active ingredient. The influence on the nebulization should also be taken into account.

Although solutions with a physiological pH are ideal, the surfaces of the lung tolerate a larger range, 3.5 to 10.0. Buffers or pH adjusting agents or vehicles can be added to adjust and stabilize the pH at a desired level. The D-HAF formulations are buffered at the pH of maximum stability of the active ingredient(s) they contain. The buffers are included to minimize any change in pH during the storage life of the drug; this can result from absorbed carbon dioxide from the air or from hydroxyl ions from a glass container. Changes in pH can affect the solubility and stability of the active ingredient(s). Consequently, it is important to minimize fluctuations in pH. The buffer system should be designed sufficient to maintain the pH throughout the expected shelf-life of the product, but with a low buffer capacity so that when the formulation is nebulized and deposited onto the linings of the lungs, the buffer system of the tears will rapidly bring the pH of the solution back to that of the linings. Low concentrations of buffer salts are used to prepare buffers of low buffer capacity.

The preparation of aqueous D-HAF formulations requires careful consideration of the need for isotonicity, a certain buffering capacity, the desired pH, the addition of antimicrobial agents and/or antioxidants, the use of viscosity-increasing agents, and the choice of appropriate packaging. The formulations are considered isotonic when the tonicity is equal to that of a 0.9% solution of sodium chloride.

Solutions that are isotonic, i.e. an amount equivalent to 0.9% NaCl is ideal for comfort and should be used when possible. There are times when hypertonic solutions are necessary therapeutically, or when the addition of an auxiliary agent required for reasons of stability supersedes the need for isotonicity. A hypotonic solution will require the addition of a substance (tonicity adjusting agent) to attain the proper tonicity range.

In some instances, the formulation is distributed or packaged in a liquid form. Alternatively, formulations for administration can be packed as a solid, obtained, for example by lyophilization of a suitable liquid formulation. The solid can be reconstituted with an appropriate carrier or diluent prior to administration.

Solutions, suspensions, or emulsions may be buffered with an effective amount of buffer necessary to maintain a pH suitable for administration. Suitable buffers are well known by those skilled in the art and some examples of useful buffers are acetate, borate, carbonate, citrate, and phosphate buffers.

Solutions, suspensions, or emulsions may also contain one or more tonicity agents to adjust the isotonic range of the formulation. Suitable tonicity agents are well known in the art and some examples include glycerin, mannitol, sorbitol, sodium chloride, and other electrolytes.

Solutions, suspensions, or emulsions may also contain one or more preservatives to prevent bacterial contamination. Suitable preservatives are known in the art, and include polyhexamethylenebiguanidine (PHMB), benzalkonium chloride (BAK), stabilized oxychloro complexes (otherwise known as PURITE®), phenylmercuric acetate, chlorobutanol, sorbic acid, chlorhexidine, benzyl alcohol, parabens, thimerosal, and mixtures thereof.

Solutions, suspensions, or emulsions may also contain one or more excipients known art, such as dispersing agents, wetting agents, and suspending agents.

In the preferred embodiments, D-HAF formulations do not contain any additives and are packaged in sterile form.

D-HAF formulations containing amniotic factors can be supplied as a clear one-part solution in a suitable container for storage at 4° C., or for storage at −20° C., or at −80° C. For example, liquid formulations in prefilled aliquots can be suitable for storage at 1-5° C., or for storage at −20° C., or at −80° C. The liquid formulation can be suitable for topical application, for example to surfaces of lungs and eyes. In other embodiments, the fluid can be supplied as a kit that can be stored at 4° C., at −20° C., or at −80° C. until needed.

### D. Kits

In some embodiments, the described compositions are provided in a kit. Typically, the compositions are prepared using a pharmaceutically acceptable carrier composed of materials that are considered safe and effective and may be administered to an individual without causing undesirable biological side effects or unwanted interactions. Typically the D-HAF composition will be in a single dose unit, for example in an ampoule ready for use with nebulizers. The D-HAF composition is in a single dose unit of about 0.1 ml to about 100.0 ml, inclusive; preferably, 1 ml to 20 ml, inclusive. In some embodiments, the D-HAF composition is in a single dose unit of about 0.5 ml, about 1.0 ml, about 2.0 ml, about 3.0 ml, about 4.0 ml, about 5.0 ml, about 6.0 ml, about 7.0 ml, about 8.0 ml, about 9.0 ml, or about 10.0 ml.

In some embodiments, the kit includes a first component containing liquid to rehydrate the dry components in a second component. For example, the first component is either water, or saline solution; and the second component is lyophilized D-HAF formulation.

In preferred embodiments, the kit includes instructions to instruct patients or practitioners as to how the dose should be used.

### III. Methods of Making

Methods of preparing the sterile de-cellularized human amniotic fluid (D-HAF) formulations have been previously described in U.S. Pat. Nos. 9,579,350; 9,884,078; 9,907,821; U.S. Patent Publication Nos. US20150025366A1; US20180142204A1; US20180311284A1; US20180140641A1.

D-HAF contains over 300 human growth factors. D-HAF is devoid of amniotic stem cells, micronized amnion membrane or chorion membrane, and/or amnion or chorion particles. The purified fluid is sterilized without the use of harsh terminal irradiation, e-beam or Ethylene Oxide (EO). In the preferred embodiment, the D-HAF is not chemical, heat, or radiation-treated. In the preferred embodiment, the process consists of separating the cells from the AF using centrifugation and utilizing a series of filtration devices to remove all remaining cells and bioburden. Each lot is tested for bioburden and is certified sterile to contain <1 harmful organisms.

### A. Preparation

The formulation is prepared from sterile human amniotic fluid obtained from a pregnant woman. The formulation is free of amnion membrane, chorion membrane, apoptotic bodies, and particulate matter, i.e. cells, large particles and other undissolvables are removed, preferably by high speed centrifugation to obtain clarified amniotic fluid. The clarified amniotic fluid is then filtered through one or more filters having a pore size of about 5 μm to about 10 μm to obtain a micron filtrate, followed by filtering the micron filtrate through one or more filters with a pore size of about 1.0 μm to obtain a second filtrate, followed by filtering the filtrate through submicron filters with the pore size of 0.45 μm or/and 0.2 μm to obtain the sterilely filtered amniotic fluid.

Those of skill in the art are well-acquainted with methods of safely and humanely obtaining samples of AF, and of the need to maintain sterility of the AF during such procedures. Suitable sources, e.g. of human AF, include AF that is obtained from patients who are undergoing amniocentesis, patients who are undergoing a Caesarean section delivery, and patients undergoing normal delivery using a specially designed receptacle to collect the fluid after rupture of membranes.

In one embodiment, the collection procedure is performed in a sterile operating room environment during an elective C-section. Typically, the woman is undergoing a pre-caesarian surgical method, and the step of obtaining the sterile human amniotic fluid includes the steps of turning on a ultrasound device to provide guidance for the process of obtaining human fluid from the woman, inserting a blunt tip needle into the amniotic sac of the woman, attaching the blunt tip needle to a three-way stopcock, connecting a Luer lock syringe to the three-way stopcock, connecting a first end of a length of sterile tubing with the three-way stopcock, and collecting sterilely the amniotic fluid through the blunt tip needle and sterile tubing into a collection container.

In this embodiment, the sterile collection container includes a pump with a suction device. The suction device is a low suction device or a spring loaded low suction device. The suction device is fluidly connected to an internal balloon. This embodiment further includes manually pumping up the internal balloon in the sterile collection container using the low suction device to allow a low-level suction and collection of the amniotic fluid.

In one embodiment, the AF collected is stored and shipped at 2-8° C. Shipments are made overnight in insulated cooler boxes with ice packs.

All processing is done under sterile conditions, in a Class 100 laminar flow hood in a clean room. As much AF as possible is separated from any solid debris. AF is transferred to sterile 500-2,000 mL containers (size depends on initial volume). Processing is performed at below 25° C. during the process.

The step of removing cells, large particles and other solids from the human amniotic fluid includes a first step of centrifuging or depth filtering the human amniotic fluid. In some embodiments, the human amniotic fluid is centrifuged at about 5,000 rpm to about 10,000 rpm for about 30 minutes to about 60 minutes. Peristaltic pumps are used to transfer the AF to clean, sterile 250 mL centrifuge bottles without over-filling the bottles. The weight of each bottle should not vary more than 2.0 grams when placed in the rotor. Use the sterile rotor sleeves over the bottles to keep them clean. Spin the bottles at 10,000 rpm for 60 minutes in the Sorvall refrigerated centrifuge. Delicately decant or pump the supernatant to a sterile container and save the pellet material. An optional second centrifugation is used when the AF is not clear of debris after the initial centrifugation. In one embodiment, the AF supernatant from the first centrifugation is transferred to sterile 50 mL centrifuge tubes which are spun at 5,000 rpm for 60 minutes. AF supernatant is decanted into a sterile container and any significant pellet volume saved.

The AF supernatant is subsequently subject to a series of filtration steps. In one embodiment about 5 μm to about 10 μm filters are used for the first filtration (pre-filtration) are cellulose ester filters, glass fiber filters, nylon capsule filters or nylon cartridge filters. In some embodiments, multiple pre-filters are used, depending on the clarity of the filtered solution. The filters with the pore size of 1.0 μm (Filtration 1.0 u) are capsule filters or cartridge filters. The filters with the pore size of 1.0 μm are poly ether sulfone, poly vinylidene fluoride or cellulose acetate membrane filters. Final filtration is carried out using filters with the pore size of 0.45 μm or 0.2 μm which are capsule filters or cartridge filters. The filters with the pore size of 0.45 μm or 0.2 μm are poly ether sulfone membrane filters, poly vinylidene fluoride or cellulose acetate membrane filters.

The sterilely filtered human amniotic fluid contains growth factors including human growth hormone, transforming growth factor beta 1, vascular endothelial growth factor, epidermal growth factor, transforming growth factor beta 3, and growth differentiation factor 11 or combinations thereof.

The sterile amniotic fluid further includes the step of filling and packaging. For example, sterile D-HAF is filled in syringes ready for application. Each shot should weigh 0.90-1.10 grams. Recalibrate pump settings if needed. Begin the fill operation using the nests of 100 Schott TopPac 1 mL syringes. Purge the air 3× from the Impro stoppering system. Stopper each nest immediately after filling using the Impro vacuum stoppering system connected to 0.2 μm filtered air. Aseptically perform at least (3) particulate counts and open media controls over the course of the run.

In a further embodiment, the filled syringes are capped with a sterile plunger. Place the syringe in a Mangar mylar pre-labeled pouch with the plunger rod towards the chevron side of the pouch. Seal the pouch with a heat sealer set to 270° F., 2.4 second dwell, 170° F. cooling temperature. Visually inspect the seals after sealing. Note that the intact syringe constitutes the primary sterile barrier of the AF product.

In yet another embodiment, the AF fluid is filled in sterile 2 ml vials with stoppers and 13 mm crimp caps as a barrier.

In some embodiments, the sterile amniotic fluid further includes the step of lyophilizing the sterile amniotic fluid to obtain a lyophilisate thereof. The method further includes irradiating the lyophilisate by e-beam irradiation or gamma ray irradiation to reinforce the sterility.

In some embodiments, the amniotic fluid from the final filtration is aseptically transferred to syringes or vials, and kept in a deep freezer at about −80° C. to about −20° C. for long term storage. The sterile amniotic fluid is dried in the vial via lyophilization in a built-in a sterile environment. The lyophilisate derived from the amniotic fluid is reconstituted with sterile water before injection or topical administration. The lyophilisate can be stored at from +4° C. to about +25° C. (room temperature).

If needed, the lyophilisate derived from amniotic fluid through lyophilization may be irradiated by e-beam irradiation or gamma ray irradiation to add another guarantee for the final sterility of the powder. Irradiation of a lyophilisate is much less denaturing for proteins and peptides than irradiating aqueous solutions, because the absence of water considerably reduces the production of reactive superoxide anions and their diffusion during irradiation. Such superoxide anions are the main cause of splitting peptide bonds and chemically modifying amino acids of protein and peptides. After lyophilization, the amniotic fluid is reconstituted by adding the initial volume of water. After gentle homogenization, the powder is quickly dissolved in about one minute.

Tools to obtain sterilely filtered human amniotic fluid from a woman, include a three-way stopcock, a sterile blunt tip needle aseptically attached to the three-way stopcock, a Luer lock syringe aseptically connected to the three-way stopcock, a sterile tubing aseptically connected to the three-way stopcock, a collection container or a collection container including a pump with suction device connected with the sterile tubing, a set of filters having the pore size of about 5 μm to about 10 μm, a set of capsule or cartridge filters having the pore size of about 1 μm, a set of capsule or cartridge filters having the pore size of about 0.45 μm or 0.2 μm, a set of sterile syringes or vials to store the sterile filtered amniotic fluid and operating instructions on using the kit to obtain sterilely filtered human amniotic fluid. The filters having the pore size of from about 5 μm to about 10 μm and the capsule or cartridge filters are made from cellulose ester, glass fiber or nylon.

The sterile collection container may include a pump with a suction device. In one aspect of this embodiment suction device may be a low suction device or spring loaded low suction device. In another aspect the suction device may be fluidly connected to an internal balloon. Further to this aspect the method includes manually pumping up the internal balloon in the sterile collection container using the low suction device to allow a low-level suction and collection of the amniotic fluid. In yet another aspect the sterile collection container may include an inlet. Further to this particular aspect the method includes connecting a second end of the tubing to the inlet of the sterile collection container. The sterile collection container may include a vent having a cap.

Utilizing the incision site immediately prior to performing the C-section and with ultrasound guidance to protect the fetus and mother provides a minimal or no risk environment for collection. Collection is achieved via a low level suction established within a collection container and/or via gravity.

Typically, high speed centrifugation filtration with 5 to 10 μm filters (low protein binding filter) is used to complete the removal of cells and large particles. Submicron filtration would then be conducted with 1 μm and 0.45 μm or/and 0.2 μm filters (low protein binding filter), two in a series connection, to remove gross contaminates. Under this condition, soluble growth factors will pass through this filter to achieve a semi-sterile condition, very low bioburden counts. If under a strict aseptic operation condition, a 10⁻³ sterility assurance level is achieved. A 10⁻⁶ sterility assurance level can be achieved by submicron filtration with a 0.22 μm filter (low protein binding filter) at the end and sterile packaging to achieve a sterile product. One would monitor the filtrate after each filtration step to determine which components were removed and then to determine which process to use to achieve the desirable product.

One may use membrane filters including or made of hydrophilic polyethersulphone (PES) to filter protein solutions. Filter disks for small volumes and different sizes of cartridges for larger volumes such 1 litre and more. Hydrophobic membranes like PTFE which are designed for liquids devoid of proteins should not be used. Start with centrifugation at 5000 to 8000 rpm for at least 30 minutes. Next, the supernatant is filtered with a prefilter to remove residual protein aggregates and precipitates in suspension (AP20 can be used). If one directly uses a 0.6/0.2 μm filter, after prefiltration, one may experience slow filtration rates and the flow may stop too quickly. It may be desirable to make intermediate filtration steps using 1.2 μm and 0.8 μm membranes. Typically, a final filtration through 0.2 μm is necessary to get the best sterility assurance level and produce a sterile amniotic fluid for injections.

### 1. Stem Cell Exosomes

In some embodiments, the exosomes present in the amniotic fluid is effective to treat one or more disclosed diseases or disorders. Thus, methods of preparing sterile de-cellularized amniotic fluid is optimized to retain most of exosomes present in the raw amniotic fluid, for example, 90%, 80%, 70%, 60%, 50%, 40%, or more than 30%.

In some embodiments, increased amount of exosomes are required for effective treatment of one or more diseases or disorders. In these cases, stem cells are induced to produce an increased amount of exosomes. Exemplary methods for induction of exosomes from stem cells include treatment of the stem cells with cytokines, treatment with liposome stimulation using one or more stimulant liposomes such as neutral or cationic liposomes (Emam S E et al., Biol Pharm Bull. 2018;41(5):733-742), or other physical and/or biological methods previously described (Phan J et al., J Extracell Vesicles. 2018; 7(1): 1522236).

Generally, methods of isolating exosomes are known including one or more of differential ultracentrifugation-based techniques, size-based techniques, immunoaffinity capture-based techniques, exosome precipitation, and microfluidics-based techniques (Li P et al., Theranostics. 2017; 7(3): 789-804).

In some embodiments, the amniotic fluid formulations further include exogenous exosomes generated ex vivo from amniotic fluid MSCs, or derived from MSCs of other sources.

### B. Storage

The final filtrate can be stored in frozen condition at about −20° C. to about −80° C. for long-term storage. In addition, the sterilely filtered amniotic fluid may be distributed in vials equipped with special rubber stoppers for sterile lyophilization.

The lyophilization is carried out in a sterile environment. The rubber stoppers on the vials are then automatically pushed down in the freeze dryer to definitively close them. Then an aluminum cap is sealed on each vial to protect its sterile content. In such a lyophilized state, the amniotic fluid may be stored at +4° C. or room temperature for at least one year without decrease of its biological activity. For its medical use, the sterile amniotic fluid may be reconstituted by adding the initial volume of sterile water to the powder in order to restore a transparent and homogeneous physiological liquid.

The decellularization and purification process protects the growth factors and other biological components of amniotic fluid from chemical and enzymatic degradation. Molecules contained within the fluid are stabilized against degradation, avoiding the need for chemical or physical modification to maintain the biological activity of the molecules over extended periods of time. Therefore, D-HAF prepared according to the described methods can be stored for long periods of time, allowing for a broad range of application methods, including distribution and storage as aerosols, solutions, powders, etc.

In some embodiments, the sterile D-HAF is refrigerated at about 1° C. to about 10° C. for long-term storage. In a further embodiment, the sterile D-HAF is refrigerated at 4° C. for up to 12 months and more. In yet another embodiment, the sterile D-HAF is stored at room temperature for over a week, 2 weeks, 3 weeks, a month, 2 months, 3 months, 6 months, up to 12 months and more while retain most biologically active components, preferably comparable to those in the D-HAF refrigerated at about 1° C. to about 10° C. for a similar duration. For example, fluids purified according to the described methods retain the biological properties of the component molecules over extended periods of storage, ideally without the need for freeze/thawing.

Preferably, the long-term storage does not reduce the quantity of the total soluble proteins or factors present in the D-HAF. For some embodiments, the total soluble proteins retained after long-term storage in frozen, refrigerated or room temperature conditions is about 10%, 20%, 30%, 40%, 50%, 60%, 70%, 80%, or 90% of the fresh D-HAF.

The protein quantities remaining soluble in the D-HAF after a period of storage is assessed by common protein quantification methods such as bicinchoninic acid (BCA) assay, Bradford assay, Lowry assay, and ultraviolet absorption (at 280 nm). To quantify individual proteins, high-throughput methods such as high-density screening arrays (RayBiotech, Norcross Ga.) are used.

Further, the storage does not reduce, prevent or otherwise alter the biological activity of any one or more of the amniotic factors of the DHAF. For example, in some embodiments, the biological activity of one or more amniotic factors is retained throughout storage for extended periods of time. The activity of any one or more of the amniotic growth factors of the stored product can be assessed as a % compared to that of the fresh (raw) product, or compared to the D-HAF prior to storage. Therefore, in some embodiments, little or no statistically significant changes in the biological activity of the amniotic factors are observed when using D-HAF stored at 4° C. or at room temperature for up to a day, 2 days, 3 days, 4 days, 5 days, 6 days, up to one week, up to 2 weeks, up to 3 weeks, up to 4 weeks, up to one month, up to 2 months, up to 3 months, up to 4 months, up to 5 months, up to 6 months or more than 6 months. In other embodiments, the activity of any one of the proteins in the amniotic fluid are reduced by 50%, 40%, 30%, 20%, 10%, 5%, or less than 5% relative to the raw amniotic fluid prior to the de-cellularizing process.

In some embodiments, one or more of the growth factors is reduced after storage. For example, such growth factors include FGF7, MMP-9, GCSF, MMP-7, MMP-13, TGF-β, FGF-4, EG-VEGF and IL-8. In other embodiments, one or more of the growth factors is reduced after freeze/thawing. For example, such growth factors include FGF-21, ANG2, GDNF, FGF-19, TIMP-2, ANG-1, TGF⊕1 and M-CSF. In a preferred embodiment, one or more of the growth factors is increase compared to the fresh D-HAF, presumably due to enhanced stability at these storage conditions. Some exemplary growth factors include VEGF-α, TNF-α, and HGF. In a further embodiment, variable changes in the growth factors such as angiotensinogen, PDGF-AA, TGF-α, EGF and SCF.

In some embodiments, inflammatory markers are decreased after refrigeration at 2-8° C. or at room temperature for one, two, three or four weeks.

For example, the amount of one or more of the inflammatory proteins present in the refrigerated sample is reduced by about 1%, 5%, 10%, 20%, 30%, 40%, 50%, 60% 70%, or 80% compared to that of the fresh (raw) product. Some exemplary inflammatory markers include Eotaxin-2, IL-6, CCL18, total GRO, CXCLS, 6Ckine, and MIP-3α.

In some embodiments, inflammatory proteins are decreased after freezing. For example, the amount of one or more of the inflammatory proteins present in the sample stored in frozen condition at about −20° C. to about −80° C. is reduced by about 5%, 10%, 20%, 30%, 40%, 50%, 60% 70%, 80% or 90% compared to that of the fresh (raw) product. Some exemplary inflammatory markers include IL-1α, CXCL9, MIP-1α, and CCL5.

In other embodiments, inflammatory markers are increased after being stored for long-term either in refrigerated or frozen conditions. Some exemplary inflammatory markers include TNF-α, MIP-1β, and MCP-2.

In a preferred embodiment, anti-inflammatory molecules are not significantly decreased after being stored refrigerated or frozen for one or more days, weeks or months. In another embodiment, one or more of the anti-inflammatory molecules is decreased in D-HAF following a period of refrigeration. Some exemplary anti-inflammatory molecules include IL-8, IL-13, IL-27, CTLA-4, and IL-21. In another embodiment, one or more of the anti-inflammatory molecules is decreased in the D-HAF stored in frozen conditions. Some exemplary anti-inflammatory molecules include IL-1Ra and TGFβ1.

### IV. Methods of Use

Methods related to the compositions and formulations and their use are provided. The compositions and formulations may be prepared as pharmaceutical compositions (e.g., amniotic fluid compositions or formulations in combination with a pharmaceutically acceptable buffer, carrier, diluent or excipient) for use in the methods. For example, disclosed are methods of administration of the compositions, methods of inducing differentiation and/or proliferation of to Tregs ex vivo or in vivo, and methods of inducing or increasing the expansion and/or function of CD4+ Treg cells ex vivo or in vivo. Also disclosed are methods of inducing or increasing a population of NK cells, for example, in a subject in need thereof.

Disclosed are methods of treating a disease, disorder or condition. The method of treatment can include administering to a subject (e.g., a human patient) an effective amount of a pharmaceutical composition including the de-cellularized amniotic fluid to one or more targeted cells or tissues in the subject. For example, disclosed is a method of treating a subject having an autoimmune disease or disorder (e.g. rheumatoid arthritis) by administering to the subject an effective amount of a pharmaceutical composition including the de-cellularized amniotic fluid.

### A. Methods of Treatment

In general, the compositions and methods of treatment thereof are useful for treating one or more symptoms in a wide variety of disease and disorders associated with immune dysfunction and/or immune dysregulation as well as various infectious and malignant diseases. The compositions can also be used for treatment of other diseases, disorders and injury including neurodegenerative diseases such as Parkinson's Alzheimer's, Huntington's, etc.; inflammatory diseases, including, but not limited to ulcerative colitis, Crohn's disease, and rheumatoid arthritis.

Exemplary immune dysfunction and/or immune dysregulation include various acute and chronic inflammatory diseases, autoimmune diseases. Exemplary autoimmune diseases include Rheumatoid arthritis, Systemic lupus erythematosus (lupus), Inflammatory bowel disease (IBD), Multiple sclerosis (MS), Type 1 diabetes mellitus, Guillain-Barre syndrome, Chronic inflammatory demyelinating polyneuropathy, and Psoriasis.

In some aspects, the amniotic fluid compositions can promote immune tolerance in one or more autoimmune diseases, i.e., the ability of the immune system to prevent itself from targeting self-molecules, cells or tissues. For example, the amniotic fluid compositions are in an amount effective in promoting the generation of regulatory T cells (Tregs), suppressing proliferation of Th1 and/or Th17 cells, enhancing one or more anti-inflammatory cytokines, and/or reducing one or more pro-inflammatory cytokines, in the desired immunological environment of the disease.

The disclosed amniotic fluid formulations are suitable in treatment for diseases and disorders of many systems such as the cardiovascular, neurological, musculoskeletal and immune systems.

In some embodiments, the amniotic fluid formulations, generally with a pharmaceutically carrier are for use as a medicament for traumatic brain injury in an amount effective for significant functional recovery, increased neurite remodeling, angiogenesis and/or neurogenesis. In some embodiments, the amniotic fluid formulations are used to ameliorate liver fibrosis, exert protective effects against acute liver injury and/or enhance the anti-tumour effect against hepatocellular carcinoma. In further embodiments, the amniotic fluid formulations are used in an amount effective to inhibit the production, activities, and/or migration of inflammatory cells, for example in autoimmune diseases.

In some embodiments, the amniotic fluid formulations, generally with a pharmaceutically carrier are for use as a medicament for a treatment to inhibit inflammation in an injured tissue. Accordingly, in one aspect, the amniotic fluid formulations are provided as a medicament for treating an autoimmune condition through administration of the de-cellularized amniotic fluid including one or more MSC-derived exosomes in an amount effective to suppress an immune pathway associated with the autoimmune condition.

In some embodiments, the amniotic fluid formulations result in an increase in the proliferation or the number of anti-inflammatory cells. Generally, the improvement in exercise tolerance is observed within days, weeks, or months after the initial treatment, and exercise duration is extended up to 10%, 20%, 30%, 40%, 50%, 60%, 70%, 80%, 90%, 100%, 200%, 300%, 400%, 500% or more than 500%.

In some embodiments, the amniotic fluid formulations are effective in suppressing and inhibiting the proliferation of inflammatory immune cells, and or promoting the proliferation of immunosuppressive cells.

In some embodiments, the subject to be treated is a human. All the methods described can include the step of identifying and selecting a subject in need of treatment, or a subject who would benefit from administration with the described compositions.

### 1. Neurodegenerative Diseases

The composition and methods can also be used to deliver active agents for the treatment of a neurological or neurodegenerative disease or disorder or central nervous system disorder. The methods typically include administering the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wernicke-Korsakoff's syndrome, post-encephalitic dementia, cancer and chemotherapy-associated cognitive impairment and dementia, and depression-induced dementia and pseudodementia.

In some embodiments, the subject has a central nervous system disorder or is in need of neuroprotection. Exemplary conditions and/or subjects include, but are not limited to, subjects having had, subjects with, or subjects likely to develop or suffer from a stroke, a traumatic brain injury, a spinal cord injury, Post-Traumatic Stress syndrome, or a combination thereof.

In some embodiments, the compositions and methods are administered to a subject in need thereof in an effective amount to reduce, or prevent one or more molecular or clinical symptoms of a neurodegenerative disease, or one or more mechanisms that cause neurodegeneration. Neurodegeneration, and diseases and disorders thereof, can be caused by a genetic mutation or mutations; protein misfolding; intracellular mechanisms such as dysregulated protein degradation pathways, membrane damage, mitochondrial dysfunction, or defects in axonal transport; defects in programmed cell death mechanisms including apoptosis, autophagy, cytoplasmic cell death; and combinations thereof. More specific mechanisms common to neurodegenerative disorders include, for example, oxidative stress, mitochondrial dysfunction, excitotoxicity, inflammatory changes, iron accumulation, and/or protein aggregation.

Symptoms of neurodegenerative diseases are known in the art and vary from disease to disease. In some embodiments, the disease exhibits or is characterized by one or any combination of the following symptoms or diseases: stress, anxiety, seasonal depression, insomnia and tiredness, schizophrenia, panic attacks, melancholy, dysfunction in the regulation of appetite, insomnia, psychotic problems, epilepsy, senile dementia, various disorders resulting from normal or pathological aging, migraine, memory loss, disorders of cerebral circulation, cardiovascular pathologies, pathologies of the digestive system, fatigue due to appetite disorders, obesity, pain, psychotic disorders, diabetes, senile dementia, or sexual dysfunction. In some embodiments, the subject does not exhibit one or more of the preceding symptoms.

In some embodiments, the subject has been medically diagnosed as having a neurodegenerative disease or a condition in need of neuroprotection by exhibiting clinical (e.g., physical) symptoms of the disease. Therefore, in some embodiments, the formulations disclosed herein are administered prior to a clinical diagnosis of a disease or condition. In some embodiments, a genetic test indicates that the subject has one or more genetic mutations associated with a neurodegenerative disease or central nervous system disorder.

Neurodegenerative diseases are typically more common in aged individuals.

### 2. Autoimmune or Inflammatory Disease

Autoimmune disease happens when the body's natural defense system cannot effectively differentiate between the body's own cells and foreign cells, causing the body to mistakenly attack normal cells. There are more than 80 types of autoimmune diseases that affect a wide range of body parts. Common autoimmune diseases include rheumatoid arthritis, psoriasis, psoriatic arthritis, systemic lupus erythematosus (SLE), type 1 diabetes, inflammatory bowel disease, and thyroid diseases.

In some embodiments, the compositions can also be used for treatment of autoimmune or inflammatory disease or disorder. Exemplary autoimmune or inflammatory disease or disorder include rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

### i. Balance T Helper Cell Profile

T cells belong to a group of white blood cells known as lymphocytes, and play a central role in cell-mediated immunity. In particular, T helper cells (also known as effector T cells or Th cells) are a sub-group of lymphocytes (a type of white blood cell or leukocyte) that plays an important role in establishing and maximizing the capabilities of the immune system and in particular in activating and directing other immune cells. More particularly, Th cells are essential in determining B cell antibody class switching, in the activation and growth of cytotoxic T cells, and in maximizing bactericidal activity of phagocytes such as macrophages.

Different types of Th cells have been identified that originate in outcome of a differentiation process and are associated with a specific phenotype. Following T cell development, matured, naïve (meaning they have never been exposed to the antigen to which they can respond) T cells leave the thymus and begin to spread throughout the body. Once the naïve T cells encounter antigens throughout the body, they can differentiate into a T-helper 1 (Th1), T-helper 2(Th2), T-helper 17 (Th17) or regulatory T cell (Treg) phenotype.

Each of these Th cell types secretes cytokines, proteins or peptides that stimulate or interact with other leukocytes, including Th cells. Th1, Th2, and Th17 (inflammatory T-helper or inflammatory Th), promote inflammation responses trough secretion of pro-inflammatory cytokines, such as IL-1, IL-6, TNF-a, IL-17, IL21, IL23, and/or through activation and/or inhibition of other T cell including other Th cells (for example Th1 ell suppresses Th2 and Th17, Th2 suppresses Th1 and Th17). Tregs instead, are a component of the immune system that suppresses biological activities of other cells associated to an immune response. In particular, Tregs can secrete immunosuppressive cytokines TGF-beta and Interleukin 10, and are known to be able to limit or suppress inflammation.

An imbalance in the profile of any of the inflammatory T-helper cells is usually associated with a condition in an individual. For example, an increase profile for Th1 or Th17 leads to autoimmunity, whereas an increased Th2 cell profile leads to allergies and asthma. In particular, imbalance of Th17 cell profile has been associated with several autoimmunitary conditions. Treg cells suppress inflammation induced by all 3 other T cell lineages, and thus are crucial for preventing uncontrolled inflammation, which leads to disease. Therefore, a balanced T-helper profile is critical for health in individuals.

Compositions and methods for balancing a T-helper cell profile and in particular Th1, Th2, Th17 and Treg cell profiles, and related methods and compositions for treating or preventing an inflammatory condition associated with an imbalance of a T-helper cell profile are described. The disclosed amniotic fluid compositions can be useful for treating, preventing and/or alleviating one or more symptoms associated with an abnormal/excessive immune response, such as an auto-immune disease, a response to a vaccine or a tissue/cell transplantation.

In some embodiments, the compositions are used in an amount effective for decreasing production of pro-inflammatory cytokines, and/or promoting generation of immunosuppressive cytokines, and/or immunosuppressive phenotype of one or more immune cell types, preferably one or more of human peripheral blood mononuclear cells. In other embodiments, the compositions are used to suppress or reduce expansion of inflammatory Th1 and Th17 cells and/or promote generation of immunosuppressive Tregs. In further embodiments, the compositions are used to suppress pro-inflammatory and promote immunosuppressive properties of one or more immune cells involved in the one or more immunological conditions to be treated.

In some embodiments, the compositions and formulations are used for therapeutic immunosuppression strategies useful in the treatment of inflammatory diseases or disorders, autoimmune diseases or disorders, inducing or increase graft tolerance, treating graft rejection, and treating allergies and other ailments with symptoms that can be reduced or ameliorated by regulating the activity of T cells (Th1, Th17, and/or Tregs), NK cells, antigen-presenting cells, or combinations thereof. In some embodiments, the methods can reduce autoantibodies (e.g., anti-dsDNA autoantibodies) production in a subject administered with the compositions.

In some embodiments, the compositions and formulations are used for modulating an immune response in a subject in need thereof by administering an effective amount of the compositions to reduce activation, proliferation and/or generation of one or more pro-inflammatory cells, and/or enhance activation, proliferation and/or generation of one or more suppressive immune cells are provided. In some embodiments, the pro-inflammatory cells are T helper type 1 (Th1) cells, T helper type 17 (Th17) cells, or both. In further embodiments, the suppressive immune cells are regulatory T cells (Tregs).

The methods are effective in treating one or more diseases or disorders selected from the group consisting of inflammatory diseases, autoimmune disorders, and transplant rejection.

In some embodiments, the therapeutic formulation provided is capable of inducing a state of anergy or immune tolerance by increasing the total number or proliferation of regulatory T cells (such as Treg), or reducing the total number or proliferation of the pro-inflammatory T cells (such as Th1 and Th17), or increase the ratio of the level of regulatory T cells (such as Treg) to pro-inflammatory T cells (such as Th1 and Th17). Thus, in some aspects, the amniotic fluid compositions are formulated for inducing anergy or tolerance by increasing Treg levels, or decrease pro-inflammatory T cell levels, or both. In other embodiments, the amniotic fluid formulations can promote suppressor/regulatory cells to cause anergy or clonal deletion of T cells by secreting inhibitory cytokines or inducing T cell apoptosis in the periphery.

In further embodiments, the compositions and formulations can attenuate production of inflammatory cytokines and/or induce the production of anti-inflammatory cytokines. Exemplary inflammatory cytokines include TNF-α, IL-1, IL-6, IL-12, IL-17, IL21, and IL23.

### B. Dosages and Dosing Regimens

Dosage and dosing regimens are dependent on the severity and location of the disorder or injury and/or methods of administration, and is known to those skilled in the art.

The formulation disclosed will be tailored to the individual subject, the nature of the condition to be treated in the subject, and generally, the judgment of the attending practitioner. In one embodiments, the formulation is in a dosage between about 0.1 ml and about 100 ml, inclusive; or between about 0.1 ml and 1 ml, inclusive; or between about 1 ml and about 10 ml, inclusive; or between about 10 ml and about 50 ml, inclusive. In yet another embodiment, the formulation is combined with any amount of between about between about 0.1 ml and about 100 ml, inclusive; or between about 0.1 ml and 1 ml, inclusive; or between about 1 ml and about 10 ml, inclusive; or between about 10 ml and about 50 ml, inclusive, of sterile water, or saline solution.

Typically, amniotic fluid formulations are packaged into sterile dosage units which can be stored and distributed for use by attending physicians. Lyophilized or fluid formulations can be in the form of sterile packaged ampoule ready for use. A filled ampoule contains a formulation of D-HAF. This is generally in a pharmaceutically acceptable carrier and buffered for human use to a pH of about 3.5-10.0, preferably about pH 6.0-7.5. In some embodiments, the formulations are free of preservative where preservatives may exert opposite effects to that required by the formulation. Water or saline solution is used to provide the carrier.

Generally, volumes used here refer to freshly processed, sterile de-cellularized human amniotic fluid i.e. 1× strength without any dilution or concentration. In some embodiments, the volumes for use with a nebulizer need to be adjusted/increased to match the amount of active ingredients in the amniotic fluid formations, in particular if the formulations were stored for a long period of time where active ingredients (amniotic factors) have deteriorated over time. In some cases, where lyophilized amniotic fluid formulations are used, these volumes refer to the volume of fluid when the lyophilized powder is reconstituted with the initial volume of sterile water i.e. 1× strength.

The sterile amniotic fluid formulation can be administered in concentrated form, diluted with sterile water, saline or buffer, preferably in the form of aerosol. It can include additional therapeutic, prophylactic or diagnostic agent, either mixed in with the formulations, or in separate containers to be used in conjunction with, subsequent to, or alternation with treatment with amniotic fluid formulation of the disclosure. The efficacy is determined by physician evaluations, patient self-evaluations, imaging studies and quality of life evaluations. The formulations can be administered locally or systemically.

One or more tonicity adjusting agents may be added to provide the desired ionic strength. Tonicity-adjusting agents for use include those which display no or only negligible pharmacological activity after administration. Both inorganic and organic tonicity adjusting agents may be used. Compositions can also include excipients and/or additives. Examples of these are surfactants, stabilizers, complexing agents, antioxidants, or preservatives which prolong the duration of use of the finished pharmaceutical formulation, flavorings, vitamins, or other additives known in the art. Complexing agents include, but are not limited to, ethylenediaminetetraacetic acid (EDTA) or a salt thereof, such as the disodium salt, citric acid, nitrilotriacetic acid and the salts thereof. In one embodiment, the complexing agent is EDTA. Preservatives include, but are not limited to, those that protect the solution from contamination with pathogenic particles, including benzalkonium chloride or benzoic acid, or benzoates such as sodium benzoate. Antioxidants include, but are not limited to, vitamins, provitamins, ascorbic acid, vitamin E or salts or esters thereof. No preservatives are preferred.

In some embodiments, lyophilized D-HAF formulations are preferred. In some embodiments, the lyophilized D-HAF is reconstituted by adding the initial volume of water. In other embodiments, the formulation is further diluted to from about 1% to about 99% of the reconstituted D-HAF. The refrigerated formulation is readily diluted to from about 1% to about 99% of the original D-HAF to a desired concentration for applications. p In other embodiments, the final formulation is prepared as a much more concentrated solution depending on the need of application. For example, to minimize the amount of time patient needs to be confined to a nebulizer, a concentrated formulation is used to deliver the same effective dosage in a shorter period. In one embodiment, the lyophilized D-HAF is reconstituted by adding half of the initial volume of water to achieve twice as concentration solutions of all amniotic factors. In a further embodiment, the lyophilized D-HAF is reconstituted by adding 10% of the initial volume of water to achieve 10-fold more concentrated solutions of the amniotic factors for application. In some embodiments, the refrigerated D-HAF can be used to reconstitute the lyophilized D-HAF in order to obtain a more concentrated solution.

The D-HAF formulations can be administered as frequently as necessary and appropriate. The frequency generally depends on the severity of the disorder or tissue damage, and the responsiveness of the target tissues to the treatment with D-HAF formulations. In some embodiments, the D-HAF formulations are administered on once-a-week basis. In other embodiments, the D-HAF formulations are administered on one-a-month basis. In some embodiments, the administration routine can changed based on the practitioners assessment of the patient after the prior treatment.

### C. Controls

The effect of amniotic fluid formulations can be compared to a control.

Suitable controls are known in the art and include, for example, an untreated subject, or a placebo-treated subject. In some embodiments, an untreated control subject suffers from, the same disease or condition as the treated subject e.g. inflammatory bowel disease.

The present invention will be further understood by reference to the following non-limiting examples.

### EXAMPLES

### Example 1

### Preparation and Analysis of AF and Exo-d-MAPPS samples Materials and Methods

### Preparation of AF and Exo-d-MAPPS Samples

Amniocentesis was performed at 15 to 18 weeks gestational age of healthy patients. Blood samples were given by the patients prior to or at the time of collection and were tested by laboratories certified under the Clinical Laboratory Improvement Amendments (CLIA) and were found negative using United States (U.S) Food and Drug Administration (FDA) licensed tests for detection of at minimum: Hepatitis B Virus, Hepatitis C Virus, Human

Immunodeficiency Virus Types 1/2, Treponema Pallidum. AF samples were obtained with patient consent as well as institutional ethical approval, as previously described (Miron P M. Curr Protoc Hum Genet. 2018:e62). Exo-d-MAPPS samples were engineered as AF-derived sterile product containing AF-MSC-Exos, manufactured under current Good Manufacturing Practices (cGMP), regulated and reviewed by the FDA (Harrell C R, et al., Ser J of Exp Clin Res. 2017;1:1). Sterile de-cellularized amniotic fluid or Exo-d-MAPPS sample incorporate Regenerative Processing Plant's (RPP) proprietary patented sterilization process to provide safe sterile product (see U.S. Pat. Nos. 9,579,350; 9,884,078; 9,907,821). Exo-d-MAPPS samples, used in this study, were manufactured under specific conditions in order to be applicable for bioavailability testing and for different therapeutic use.

Isolation of pbMNCs

The serum samples (2 ml) were obtained from healthy volunteers at the Center for Molecular Medicine and Stem Cell Research Faculty of Medical Sciences University of Kragujevac, and pbMNCs were isolated by the use of Histopaque (Sigma-Aldrich, Munich, Germany) density gradient centrifugation. Briefly, the serum was diluted by equal volume of Dulbecco's modified Eagle's medium (DMEM) supplemented with 10% fetal bovine serum, 2 mmol/L of L-glutamine, 1 mmol/L penicillin-streptomycin, and 1 mmol/L of mixed nonessential amino acids, (Sigma Aldrich, Munich, Germany). Heparinized peripheral blood (10 ml) was centrifuged at 400 g for 10 min to separate plasma and cells. Lymphocyte separation liquid (3 ml) was filled into a 10-mL centrifuge tube. After 10 min, the mononuclear cell layer was transferred to a sterile tube by a fresh sterile pipette (capillary tube), gently mixed with five volumes of DMEM and centrifuged at 2700 r/min for 20 min, then washed with DMEM twice. After the supernatant was discarded, the cells were resuspended in DMEM containing 10% fetal bovine serum (Gibco, United States) for lymphocyte count. Then the cells suspensions were diluted to 1×10⁶ cells/ml for further in vitro experiments.

Activation of pbMNCs

After isolation, pbMNCs were plated in a 24-well plate (1×10⁶ cells per well) and subsequently primed with 10 ng/ml Lipopolysaccharides (LPS) (Meng F, et al., J Exp Med. 1997;185:1661-70), or 5 μg/ml Concanavalin A (Con A)-potent activator of T cells (Volarevic V, et al., Hepatology. 2012;55:1954-1964), or 100 ng/ml of alpha galactosyl ceramide (α-GalCer)-selective stimulator of NKT cells (Gazdic M, et al., J Tissue Eng Regen Med. 2018;12:e1173-e1185; Volarevic V, et al., Hepatology. 2012;55:1954-1964; Pejnovic N N, et al., Diabetes. 2013;62:1932-1944). Isolated pbMNCs were cultured in complete DMEM for 48 h in the presence or in the absence of Exo-d-MAPPS and AF. After 48 hours of culture, activated pbMNCs were harvested for ELISA assay or flow cytometry.

### Measurement of Cytokines in Supernatants

The ELISA assay was conducted according to the handbook provided by the ELISA kit (R&D Systems Minneapolis, Minn. for IL-12, IL-17 and IL-10; BD Biosciences San Diego, Calif. for IFN-γ). The optical density of each sample at 450 nm was detected by an ELISA microplate reader (Zenyth, 3100) and the concentrations of IL-12, IL-17, IFN-γ and IL-10 levels in supernatants were determined.

Flow Cytometry Analysis of pbMNCs

To detect the cell surface expression of a variety of molecules, isolated pbMNCs were analyzed by flow cytometry (FACS) using standard staining methods (Volarevic V, et al., Cells. 2019;8). Briefly, the prepared cell suspension fluid (1 ml) centrifuged at 250 g for 5 min and rinsed twice with suspension fluid. The supernatant was discarded and cells were suspended with PBS to 10 μl, adding human CD14, CD56, HLA-DR and CD4 antibody conjugated with fluorescein isothiocyanate (FITC; BD Biosciences, Franklin Lakes, N.J.), phycoerythrin (PE; BD Biosciences) or allophycocyanin (APC; BD Biosciences or isotype-matched controls (BD Pharmingen/BioLegend) (about 1.25 μg, suggested by the manual) respectively, and incubated at 4° C. in the dark for 30 min. Then, the cell suspension was supplemented with 2 ml PBS, centrifuged at 250 g 5 minutes, and washed with suspension fluid followed by staining with flow cytometry staining buffer. For the intracellular staining, cells were previously stimulated with phorbol myristate acetate (PMA) and ionomycin for 4 h at 37° C. with the addition of 1 μg/mL Golgi plug. Intracellular staining for forkhead box P3 (Foxp3), IL-10, tumor necrosis factor alpha (TNF-α), IL-17, interferon gamma (IFN-γ) was performed using the BD Bioscience fixation/permeabilization buffer kit. Flow cytometric analysis was conducted on a BD Biosciences FACSCalibur and analyzed by using the flowing software analysis program.

### Statistical Analysis

Results were analyzed using the Student's t test. All data in this study were expressed as the mean±standard error of the mean (SEM). Values of p<0.05 were considered as statistically significant.

### Results

Exo-d-MAPPS Attenuated Production of Inflammatory Cytokines and Promotes Generation of Immunosuppressive Phenotype in LPS-Primed pbMNCs

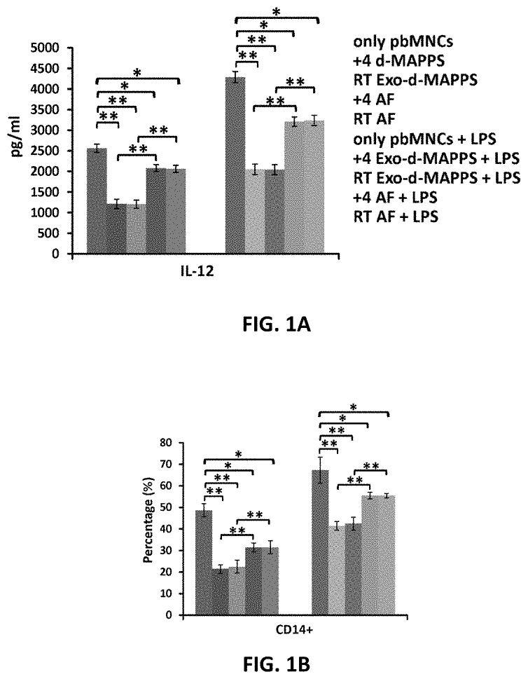

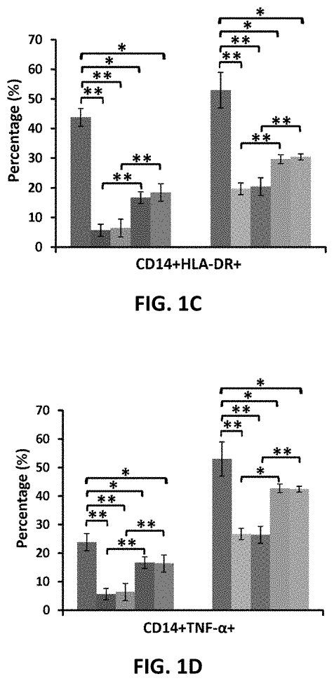

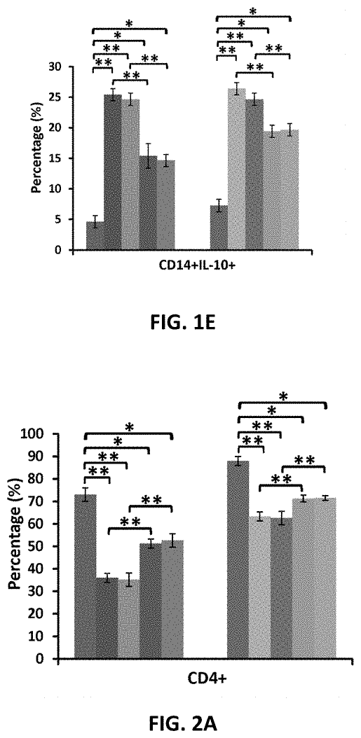

LPS significantly enhanced production of inflammatory IL-12 in pbMNCs (FIG. 1A). Exo-d-MAPPS significantly attenuated concentration of IL-12 in the supernatants of LPS-primed pbMNCs (FIG. 1A). Importantly, both room temperature (RT) and fridge (4° C.) stored Exo-d-MAPPS suppressed production of IL-12 more efficiently than RT and 4° C. stored AF (FIG. 1A). Since LPS mainly activates CD14-expressing macrophages, we analyzed whether Exo-d-MAPPS affected percentage of this cell population. As it is shown in FIG. 1B, the percentage of LPS-primed pbMNCs that expresses CD14 was significantly lower after Exo-d-MAPPS treatment. Additionally, Exo-d-MAPPS down-regulated expression of HLA-DR molecule and production of inflammatory TNF-α in CD14-expressing pbMNCs (FIGS. 1C and 1D). In similar manner as it was observed in the attenuation of IL-12 production, Exo-d-MAPPS-treated LPS-primed CD14+pbMNCs produced lower amount of TNF-α than AF-treated LPS-primed CD14+pbMNCs (FIGS. 1C and 1D). In line with these findings, Exo-d-MAPPS treatment induced generation of immunosuppressive phenotype in LPS-primed CD14+pbMNCs (FIG. 1E). Significantly higher percentage of IL-10-producing CD14+ cells and significantly higher concentration of IL-10 was observed in supernatants of Exo-d-MAPPS treated LPS-primed pbMNCs than in the supernatants of AF-treated LPS-primed pbMNCs (FIG. 1E)

Exo-d-MAPPS Treatment Reduced Expansion of Inflammatory Th1 and Th17 Cells and Promoted Generation of Immunosuppressive Tregs in the Population of Con A-Primed pbMNCs

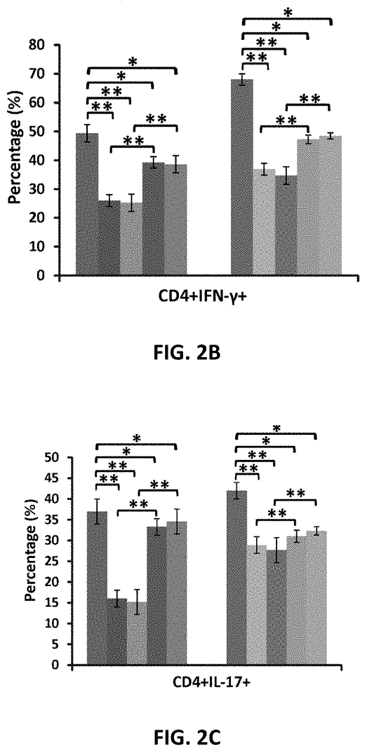

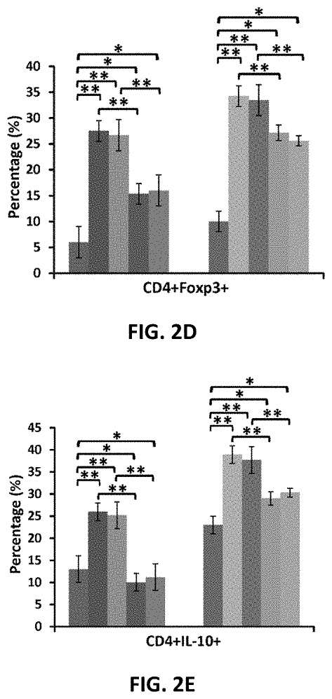

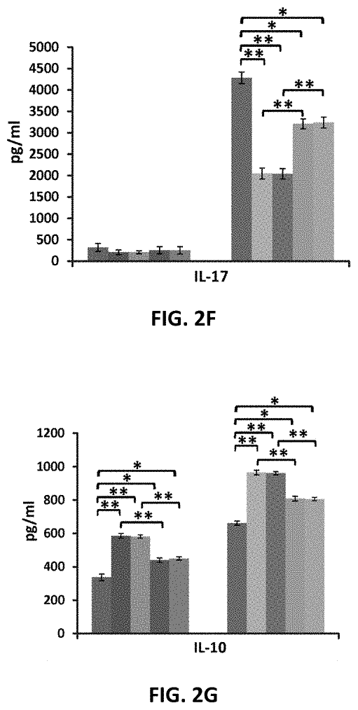

Con A treatment induced expansion of CD4+ cells and, particularly, inflammatory, IFN-γ-producing Th1 and IL-17-producing Th17 CD4+ T cells within the population of pbMNCs (FIGS. 2A-2C). Exo-d-MAPPS significantly attenuated expansion of CD4+ cells and alleviated production of IFN-γ and IL-17 in Con A-primed CD4+ T cells (FIGS. 2A-2C). Importantly, treatment with either RT or 4° C. stored Exo-d-MAPPS more efficiently reduced expansion of inflammatory Th1 and Th17 cells than AF (FIGS. 2A-2C), indicating superior immunosuppressive properties of Exo-d-MAPPS over AF. Additionally, Exo-d-MAPPS promoted generation of immunosuppressive phenotype in CD4-expressing pbMNCs, as evidenced by higher percentage of FoxP3-expressing and IL-10-producing CD4+ cells in the population of Exo-d-MAPPS-treated Con A-primed pbMNCs compared to Con A-only and AF+Con A-treated pbMNCs (FIGS. 2D and 2E). In line with these findings, significantly lower concentration of IL-17 and higher concentration of IL-10 were noticed in the supernatants of Exo-d-MAPPS+Con A-treated pbMNCs compared to Con A-only and AF+Con A-treated pbMNCs (FIGS. 2F and 2G).

Exo-d-MAPPS Treatment Suppressed Pro-Inflammatory and Promoted Anti-Inflammatory Properties of α-GalCer-Primed pbMNCs

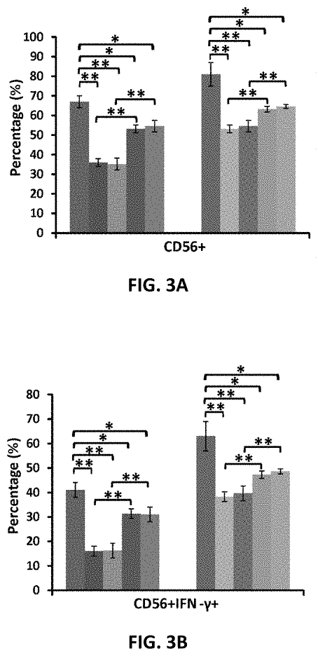

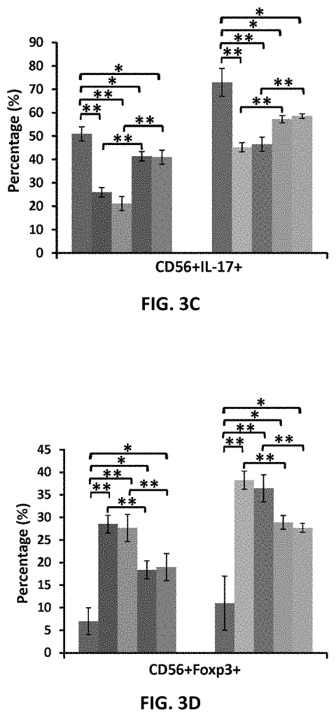

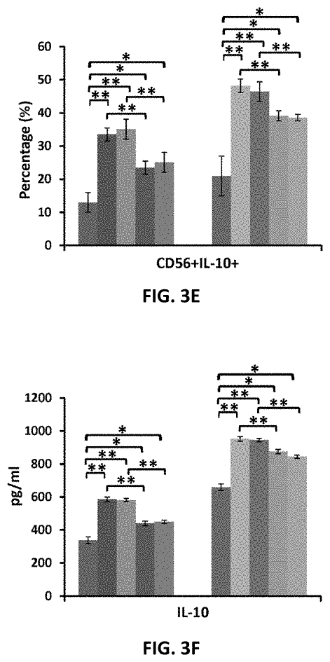

As it is shown in FIG. 3A, α-GalCer treatment stimulated expansion of inflammatory, IFN-γ-producing and IL-17-producing CD56-expressing cells within population of pbMNCs. Both RT and 4° C. stored Exo-d-MAPPS more efficiently reduced proliferation of IFN-γ-producing and IL-17-producing CD56+ cells than RT or 4° C. stored AF (FIGS. 3B and 3C). In similar manner as it was observed with Con A-primed pbMNCs, Exo-d-MAPPS treatment promoted generation of immunosuppressive phenotype in α-GalCer-activated pbMNCs (FIGS. 3D and 3E). Significantly higher percentage of FoxP3-expressing and IL-10-producing CD56+cells were observed in the population of α-GalCer+Exo-d-MAPPS-treated pbMNCs than in α-GalCer-only and α-GalCer+AF treated pbMNCs (FIGS. 3D and 3E). In line with these findings, significantly lower concentration of immunosuppressive IL-10 was measured in the supernatants of α-GalCer+Exo-d-MAPPS-treated pbMNCs than in the supernatants of α-GalCer-only and α-GalCer+AF-treated pbMNCs (FIG. 3F).

A large number of experimental and clinical evidence suggested that

MSCs, due to their immunomodulatory properties, should be considered as new therapeutic agents for the treatment of autoimmune and incurable inflammatory diseases (Rad F, et al., Mol Biol Rep. 2019;46:1533-1549; Harrell C R, et al., Stem Cells Int. 2019;2019:4236973; Markovic B S, et al., Stem Cell Rev. 2018;14:153-165; Gazdic M, et al., Int J Biol Sci. 2017;13:1109-1117). Despite of promising results observed after autologous and allogeneic transplantation of MSCs, safety issues regarding MSCs-based therapy are still a matter of debate (Volarevic V, et al., Int J Med Sci. 2018;15:36-45). Due to their multipotency, MSCs may spontaneously differentiate into the undesired cell type, particularly osteocytes and chondrocytes. Although malignant transformation of transplanted MSCs has not been validated, high proliferation rate and capacity for self-renewal indicate possible risk of mutations which may result in tumor development and therefore long-term follow up of patients that received MSCs is required. Several studies revealed that despite of low engraftment rate therapeutic effects of MSCs remained long after transplantation, suggesting that beneficial effects of MSC-based therapy were relied on the activity of MSC-sourced factors rather than on the differentiation of engrafted MSCs (Gnecchi M, et al., Methods Molecular Biol (Clifton, N.J.). 2016;1416:123-146; Liang X, et al., Cell Transplant. 2014;23:1045-1059; Wang A, et al., Stem Cells Transl Med. 2015;4:659-669). Recently published studies indicated that MSC-derived immunosuppressive factors might be delivered to the target immune cells within MSC-Exos which, due to their nano-sized dimension and lipid envelope, easily avoid biological barriers in the body (Rad F, et al., Mol Biol Rep. 2019; 46:1533-1549). In line with these findings, it is now demonstrated that Exo-d-MAPPS, soluble product which contains a broad number of MSC-derived immunomodulatory factors (Harrell CR, et al., Ser J of Exp Clin Res. 2017;1:1), efficiently suppress inflammatory properties of pbMNCs and should be considered as potentially new agent for the treatment of acute and chronic inflammatory diseases.

Capacity of LPS-primed CD14-expressing monocytes for the production of inflammatory cytokines (TNF-α and IL-12) was significantly attenuated by Exo-d-MAPPS treatment (FIGS. 1A and 1D). CD14 is a LPS-binding protein, expressed on the membrane of macrophages and DCs, playing crucial role in the immune recognition of the microbial cell wall components from Gram-negative bacteria (Zamani F, et al., Adv Pharm Bull. 2013; 3:329-332). Cross-talk between LPS-activated, CD14-expressing monocytes and IFN-y-producing CD4+ Th1 cells has crucially important role in the pathogenesis of many autoimmune and chronic inflammatory diseases (diabetes mellitus, multiple sclerosis, Crohn's disease, etc.) (Xu Y, et al., Immunology. 2016;149:157-171; Hirahara K, et al., Int Immunol. 2016;28:163-171). CD14-expressing macrophages and DCs, through the production of “pro-Th1 cytokines” (TNF-α and IL-12), induce generation of IFN-y-producing CD4+ Th1 effector cells, which in turn, through the secretion of IFN-γ promote phagocytic activity and capacity for antigen presentation of CD14-expressing monocytes. Exo-d-MAPPS treatment resulted in attenuated production of TNF-α and IL-12 in activated CD14-expressing monocytes (FIGS. 1A and 1D) and alleviated production of IFN-γ in activated CD4+ cells (FIG. 2B), indicating its capacity for suppression of CD4+Th1:macrophage crosstalk and therapeutic potential for the treatment of chronic inflammatory diseases. Additionally, Exo-d-MAPPS significantly attenuated production of IL-17 in activated CD4+ T cells (FIG. 2F) and inhibited expansion of Th17 cells (FIG. 2C). IL-17 and Th17 cells have important pathogenic role in chronic organ-specific and systemic inflammatory disorders and, therefore, alleviation of IL-17-driven immune response has been responsible for beneficial effects of MSCs and MSC-derived secretome in the therapy of liver fibrosis, multiple sclerosis, systemic lupus erythematosus and rheumatoid arthritis (Mills K H. Eur J Immunol. 2008;38:2636-2649; Bunte K, et al., Int J Mol Sci. 2019;20; Chehimi M, et al., J Clin Med. 2017;6; Harrell C R, et al., Adv Exp Med Biol. 2018;1089:47-57). Several lines of evidence indicated that MSCs in IDO/Kynurenine, TGF-β and PGE2-dependent manner increased Tregs/Th17 ratio by promoting generation of immunosuppressive Tregs during the differentiation process of Th17 cells (Harrell C R, et al., Adv Exp Med Biol. 2018;1089:47-57; Wang D, et al., Cell Mol Immunol. 2017;14:423-431; Luz-Crawford P, et al., Stem Cell Res Ther. 2013;4:65). In line with these findings, Exo-d-MAPPS, that contains MSC-derived Treg-promoting factors (Harrell C R, et al., Ser J of Exp Clin Res. 2017;1:1), concomitantly suppressed proliferation of Th17 cells and induced enhanced expansion of IL-10-producing Tregs (FIGS. 2C and 2D).

Similarly, Exo-d-MAPPS induced expansion of FoxP3-expresing and IL-10-producing CD56+ cells and suppressed proliferation of inflammatory IFN-γ and IL-17 α-GalCer-primed pbMNCs. Exo-d-MAPPS is potentially a new remedy for the attenuation of NKT cell-dependent acute liver failure.

Since there was not significantly difference in immunomodulatory potential of RT and 4° C. Exo-d-MAPPS samples, Exo-d-MAPPS can be used as a soluble product either stored at RT or at 4° C. Importantly, although Exo-d-MAPPS is AF-MSC-derived product, Exo-d-MAPPS suppressed generation of inflammatory phenotype in pbMNCs significantly better than the raw AF. Exo-d-MAPPS contains AF-MSC-derived Exos, extracellular vesicles which diameter is smaller than 100 nm and do not contain apoptotic bodies (Harrell C R, et al., Ser J of Exp Clin Res. 2017;1:1). During the production of Exo-d-MAPPS, due to the centrifugation and filtration, large extracellular vesicles (with diameter bigger than 100 nm), including apoptotic bodies and microvesicles were removed from the secretome (Harrell C R, et al., Ser J of Exp Clin Res. 2017;1:1). Since apoptotic bodies have been implicated in inducing activation of inflammatory cascade in immune cells (Tannetta D, et al., Cell Mol Immunol. 2014;11:548-563), the absence of these apoptotic bodies in Exo-d-MAPPS samples resulted in their better immunosuppressive potential compared to the raw AF samples.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. A pharmaceutical composition comprising de-cellularized amniotic fluid (D-HAF) and one or more pharmaceutically acceptable excipients.

2. The pharmaceutical composition of claim 1, wherein the D-HAF is devoid of amniotic cells, micronized amnion membrane and chorion membrane particles.

3. The pharmaceutical composition of claim 1 further comprising one or more agents selected from the group consisting of neuroprotective agents, antimicrobial agents, local anesthetics, antioxidants, anti-inflammatory agents, growth factors, immunosuppressant agents, anti-allergic agents, and combinations thereof.

4. The pharmaceutical composition of claim 1 further comprising one or more exosomes generated ex vivo from mesenchymal stem cells.

5. The pharmaceutical composition of claim 4 where in the mesenchymal stem cells are amniotic fluid mesenchymal stem cells.

6. A method of modulating an immune response in a subject in need thereof comprising administering an effective amount of the pharmaceutical composition of claim 1 to reduce activation, proliferation and/or generation of one or more pro-inflammatory cells, and/or enhance activation, proliferation and/or generation of one or more suppressive immune cells.

7. The method of claim 6, wherein the one or more pro-inflammatory cells are T helper type 1 (Th1) cells, T helper type 17 (Th17) cells, or both.

8. The method of claim 6, wherein the pharmaceutical composition is in an effective amount to reduce the frequency and/or number of Th1, Th17, or both.

9. The method of claim 6, wherein the one or more suppressive immune cells are regulatory T cells (Tregs).

10. The method of claim 6, wherein the pharmaceutical composition is in an effective amount to increase the frequency and/or number of Tregs.

11. The method of claim 6, wherein the immune response is one associated with one or more diseases or disorders selected from the group consisting of inflammatory diseases, autoimmune disorders, and transplant rejection.

12. The method of claim 11, wherein the autoimmune disorders are selected from the group consisting of rheumatoid arthritis, systemic lupus erythematosus, alopecia areata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

13. A method of increasing a ratio of the level of endogenous regulatory T (Treg) cells to the level of endogenous pro-inflammatory T cells in a subject in need thereof for treating or alleviating one or more symptoms associated with an inflammatory disease, an autoimmune disorder, or transplant rejection in a subject, comprising administering to the subject an effective amount of the pharmaceutical composition of claim 1.

14. The method of claim 13, wherein endogenous pro-inflammatory T cells are T helper type 1 (Th1) cells, T helper type 17 (Th17) cells, or both.

15. A method for treating a disease or disorder associated with an elevated number of one or more pro-inflammatory cells, and/or reduced number of one or more suppressive immune cells, in a subject comprising administering to a subject an effective amount of the pharmaceutical composition of claim 1.

16. The method of claim 15, wherein the one or more pro-inflammatory cells are T helper type 1 (Th1) cells, T helper type 17 (Th17) cells, or both.

17. The method of claim 15, wherein the pharmaceutical composition is in an effective amount to reduce the frequency and/or number of Th1, Th17, or both.

18. The method of claim 15, wherein the one or more suppressive immune cells are regulatory T cells (Tregs).

19. The method of claim 15, wherein the pharmaceutical composition is in an effective amount to increase the frequency and/or number of Tregs.

20. A method for reducing Th1 and Th17 responses in a subject, the method comprising administering to the subject the pharmaceutical composition of claim 1.

21. A method for treating elevated levels of one or more cytokines selected from the group consisting of IL-1, IL-6, TNF-a, IL-17, IL21, and IL23, in a subject comprising administering to the subject the pharmaceutical composition of claim 1.

22. A method for enhancing regulatory T cell (Treg) responses in a subject comprising administering to a subject the pharmaceutical composition of claim 1.

23. A method for treating or inhibiting one or more symptoms of an inflammatory response in a subject comprising administering to the subject the pharmaceutical composition of claim 1.

24. A method for reducing or inhibiting transplant rejection in a subject in need thereof comprising administering to the subject the pharmaceutical composition of claim 1.

25. A method for treating one or more symptoms of graft versus host disease (GVHD) in a subject comprising administering to a subject who has received or will receive a transplant the pharmaceutical composition of claim 1.

26. A method for treating one or more symptoms of a stroke, a traumatic brain injury, a spinal cord injury, Post-Traumatic Stress syndrome, dementia, or a combination thereof, in a subject comprising administering to a subject an effective amount of the pharmaceutical composition of claim 1.
